(19) United States
(12) Patent Application Publication
(10) Pub. No.: US 2005/0196454 A1
(43) Pub. Date: Sep. 8, 2005

(54) Oxidatively stable magnetic metal nanoparticles prepared with copolymers containing phthalonitrile moieties, and polymer-metal complexes and their conversion to oxidatively-stable metal nanoparticles

(76) Inventors: Victor Vincent Baranauskas III, Christiansburg, VA (US); Judy Riffle, Blacksburg, VA (US)

Correspondence Address: WHITHAM, CURTIS & CHRISTOFFERSON, P.C., 11491 SUNSET HILLS ROAD, SUITE 340, RESTON, VA, 20190, US

(21) Appl. No.: 10/887,312

(22) Filed: Jul. 9, 2004

Related U.S. Application Data
(60) Provisional application No. 60/549,941, filed on Mar. 5, 2004.

(51) Int. Cl. H01F 1/26; A61K 9/14

(52) U.S. Cl. 424/489; 526/297; 252/62.54

(57) ABSTRACT

Oxidatively-stable, magnetic cobalt nanoparticles and other Group VIII nanoparticles are provided, and production methods. Poly(styrene-b-4-vinylphenoxyphthalonitrile) (a novel composition) or random graft copolymers containing phthalonitrile groups in the backbone are examples of compositions that may be complexed with cobalt or other Group VIII metals.

Priority is claimed based on U.S. provisional application No. 60/549,941 filed Mar. 5, 2004, titled, “Oxidatively stable magnetic metal nanoparticles prepared with copolymers containing phthalonitrile moieties.”

### STATEMENT REGARDING FUNDING

DARPA and NSF may have provided funding with regard to the invention set forth herein.

### DESCRIPTION

1. Field of the Invention

The present invention is directed to nanoparticles with magnetic properties and magnetic fluids, and complexing of polymeric materials with metals.

2. Background of the Invention

Over the years, magnetic nanoparticle technology has developed. For appreciating dimensions, it may be mentioned that a nanometer (nm) equals one billionth of a meter, or 10 angstroms. Magnetic fluids are recognized as stable colloidal suspensions of fine magnetic particles on the order of nanometers suspended within a carrier liquid. Berkovski, B., Magnetic Fluids and Applications Handbook; Begell House: New York, 1996. These magnetic fluid materials display typical properties of fluids and behave as intrinsic liquid ferromagnets that move as an entity in the direction of an applied magnetic field. Id. The foundation of magnetic fluid research was established by the work of Hess and Parker. Hess, P.; Paker, P., J. Appl. Polym. Sci., 1966, 10, 1915. Some magnetic fluids are disclosed in: U.S. Pat. App. 20030042462, published Mar. 6, 2003 (Riffle et al.) and U.S. Pat. App. 20020141942 (Riffle et al.), published Oct. 3, 2002 (both of which are incorporated herein by reference), in which magnetic fluids are provided that comprise a block copolymer stabilizer, magnetic particles and a fluid polysiloxane medium.

Magnetic fluids have been developed for commercial use in a number of applications including loud speakers, rotating shaft seals, and dampers. Berkovski, supra. Other applications being investigated include drug targeting (Yu, J.; Hafeli, U.; Li, Y.; Failing, S.; Leakakos, T.; Tapolsky, G., Fourth International Conference on the Scientific and Clinical Applications of Magnetic Carriers 2002, 16), magnetic isolation and modification of biomolecules (Cremer, A.; Reinhard, C.; Muller, S.; Gunther, G.; Kohler, M.; Koster, M.; Heckel, N.; Bierver, C.; Johnston, I.; Merkel, D.; Nolle, V.; Miltenyi, Fourth International Conference, supra, 73), and hyperthermia treatment for cancer patients (Hofer, K., Fourth International Conference, supra, 78). Magnetic drug delivery has been actively studied for at least two decades. S. R. Rudge, T. L. Kurtz, C. R. Vessely, L. G. Catterall, D. L. Williamson, “Preparation, characterization, and performance of magnetic iron-carbon composite microparticles for chemotherapy,” Biomaterials 21 (2000), 1411-1420. Regarding magnetic drug delivery, see also U.S. Pat. App. 20040086572, published May 6, 2004, “Delivery of therapeutic agent affixed to magnetic particle” (Dailey & Riffle), incorporated herein by reference. The vast majority of ferrofluid and magnetic particle research focuses on the use of iron oxides due to their oxidative stability and biocompatibility. Iron particles have been ground with charcoal to yield compositions with high magnetization, such as by a company called FeRx, as hosts for drug delivery. See U.S. Pat. No. 6,482,436, issued Nov. 19, 2002 to Volkonsky et al. (“Magnetically responsive composition); U.S. Pat. No. 6,200,547, issued Mar. 13, 2001, to Volkonsky et al. (“Magnetically responsive compositions for carrying biologically active substances and methods of production and use”); U.S. Pat. App. 20030108614, published Jun. 12, 2003, Vokonsky et al. (“Magnetically responsive composition”).

Ferrofluids are liquid dispersions of ferromagnetic or ferrimagnetic particles, usually in hydrocarbons, esters or water. Polymer or low molecular weight surfactants adsorbed or bonded on the particle surfaces can prevent agglomeration by either electrostatic or steric repulsion. The particles are often ferrimagnetic iron oxides such as magnetite or maghemite since methods for their synthesis in small sizes (e.g. ≈10 nm in diameter) are established and these materials are relatively stable against oxidation. If particle diameters are in this range, the materials can be superparamagnetic. When such fluids are placed in gradient magnetic fields, they typically respond by moving as an entity toward the direction of highest field. Berkovski, B. and Bashtovoy, V. 1996. Then, when the applied fields are removed, the magnetic moments of the particles randomize rapidly and the net magnetic moment of the fluid returns to zero.

Non-iron oxides as magnetic nanoparticles have not been much pursued. For example, conventionally, cobalt or iron particles have not been pursued for magnetic nanoparticles because such conventional cobalt or iron nanoparticles oxidize slowly in air, undesirably forming nonmagnetic oxides. Consequently, their magnetic properties decrease under ambient conditions limiting their long-term use. The development of cobalt or iron ferrofluids therefore has been limited.

One example of a conventional magnetic nanoparticle is a silica-coated nanoparticle. Undesirably, the silica coatings are required to be relatively thick, which significantly reduces the magnetic properties.

### SUMMARY OF THE INVENTION

The present invention recognizes that phthalonitrile functional groups (e.g., o-dicyanobenzene, C₆H₄(CN)₂, etc.) may be used to prepare or encapsulate nanoparticles; to make block copolymer dispersants; and/or may be introduced into copolymers used as coatings for magnetic nanoparticles.

In one preferred embodiment, the invention provides for using phthalonitrile groups to prepare or encapsulate nanoparticles.

In another preferred embodiment, the invention provides block or graft copolymer dispersants made from phthalonitrile groups.

In yet another preferred embodiment, the invention provides magnetic nanoparticles that have been coated with copolymers containing phthalonitrile groups, then pyrolyzed to form dense protective coatings, including, e.g., protective coatings that advantageously are less thick than silica coatings.

The invention also provides, in another embodiment, oxidatively stable magnetic nanoparticles.

New composition of matter on magnetic copolymer-metal nanoparticle complexes are also provided by the invention. These complexes can be pyrolyzed to form highly magnetic (greater than ˜50 emu/g) material, oxidatively stable nanoparticles. An example of such a copolymer is poly(styrene-b-4-vinylphenoxyphthalonitrile) while the metals employed are cobalt or iron.

The invention also provides new compositions of matter, including poly(styrene-b-4-vinylphenoxyphthalnitrile metal nanoparticle complexes that afford highly magnetic (greater than ˜50 emu/g) material, oxidatively stable nanoparticles.

The present invention recognizes that magnetic materials consisting of cobalt or iron metal nanoparticles have the potential for 3 to 4 times the magnetic response of the iron oxides, the vast majority of magnetic nanoparticle research having been concentrated on iron oxides. The present invention overcomes the problem that the development of cobalt and iron particles for all applications has been limited, before this invention, by the fact that metallic nanoparticles oxidize slowly in air forming nonmagnetic oxides. The present invention provides a novel material that may be used to form colloidally stable dispersions of cobalt or iron nanoparticles that may be concentrated to a solid state and pyrolyzed at 500-700° C. to afford oxidatively stable cobalt or iron nanoparticles encapsulated with dense coatings from the phthalonitrile functional group.

In one preferred embodiment, the invention provides a phthalonitrile composition, comprising poly(styrene-b-4-vinylphenoxyphthalonitrile). The poly(styrene-4-vinylphenoxyphthalonitrile) may be complexed to a Group VIII metal, such as cobalt, iron, etc.

Another preferred embodiment of the invention is Poly(styrene-b-4-vinylphenoxyphthalonitrile).

In a further preferred embodiment, the invention provides a composition selected from the group consisting of: (a) a block or graft copolymer wherein at least one block contains phthalonitrile moieties; (b) a poly(styrene-b-vinylphenoxyphthalonitrile) block copolymer comprising a polystyrene block and a polyvinylphenoxyphthalonitrile anchor block and having 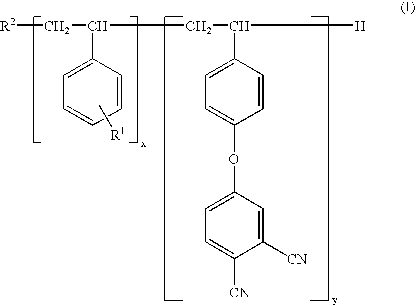
the following structure:
(wherein x and y denote the average numbers of repeating units in the block copolymer and each can vary between about 10-500; R¹ can be —H or an alkyl substituent having about 1-100 carbon atoms, aromatic substituents, or mixed alkyl aromatic substituents; R² can be any alkyl or aromatic groups derived from an initiator); or (c) a graft copolymer comprising a random sequenced mixed anchor block and PDMS tail grafts, with the structure of the graft copolymer being according to the following formula (II): 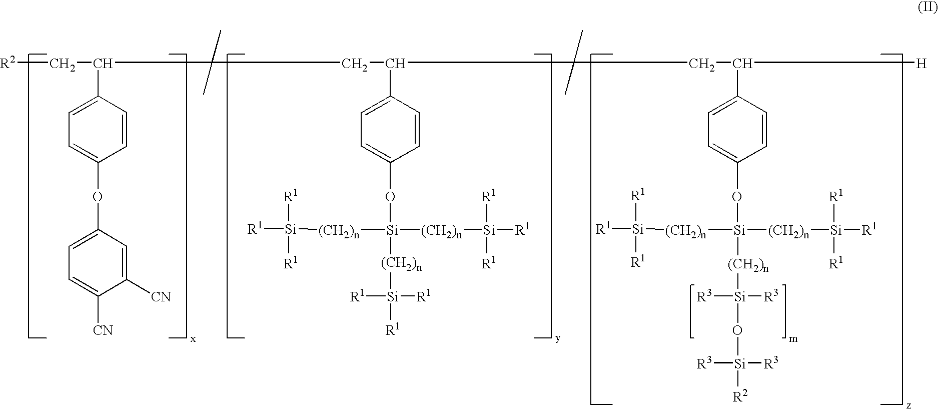
(wherein R¹ can be any group or combinations of groups that lead to a silica containing residue upon pyrolysis; R² can be any alkyl or aryl group derived from an initiator moiety; R³ can be alkyl groups containing about 1-6 carbons or aromatic groups; n is an integer of at least 1; m is an integer and represents an average number of repeating units in the polysiloxane grafts; x, y and z are each at least 1 and can be the same or different). A preferred structure for a graft copolymer in such an inventive composition is: 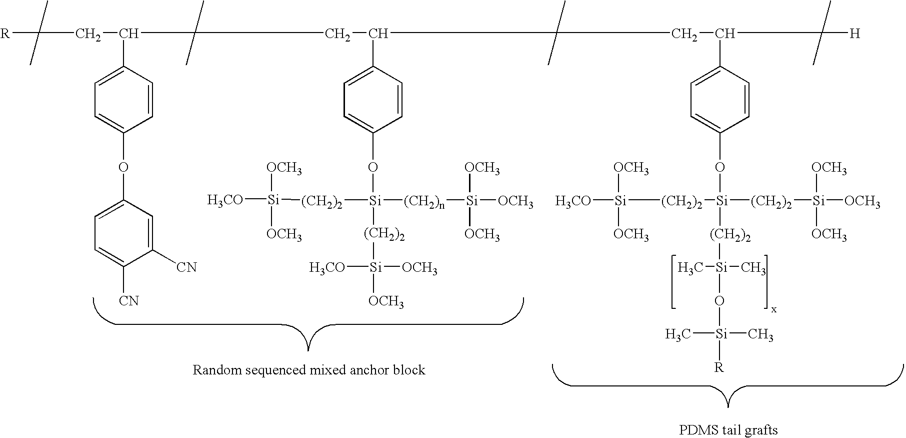
In the inventive compositions, the block or graft copolymer may be complexed to a group VIII metal (such as cobalt, iron, etc.).

The above-mentioned inventive compositions may be included in a ferrofluid (such as a cobalt ferrofluid, an iron ferrofluid, etc.); and/or may be pyrolyzed at a temperature of 300° C. or greater to produce a pyrolyzed complex. The invention further provides such pyrolyzed complexes, wherein the pyrolyzed complex has a saturation magnetization of 50 emu g⁻¹ or greater in some examples, and 90 emu g⁻¹ or greater in some examples. A pyrolyzed complex according to the invention may have a surface functionalized with functional groups, such as, e.g., functional groups selected from the group consisting of amine; isocyanate; hydroxyl; polypeptides; polyglycolides; polylactides; poly(lactide-co-glycolides); poly(ethylene oxide); poly(ethylene oxide-co-propylene oxide); other polymers; streptavidin; avidin; other proteins; polynucleotides; vitamins; steroids; and other biospecific groups.

The invention in a preferred embodiment provides a pyrolyzed matrix material, comprising: a Group VIII metal; and a carbonaceous matrix and optionally also a silica matrix, wherein the matrix comprises a pyrolysis product of a polymeric material containing phthalonitrile groups.

Another preferred embodiment of the invention provides a polymer-metal complex, comprising: a Group VIII metal (such as, e.g., Fe, Co, Ni, Ru, Rh, Pd, Os, Ir, Pt, preferably cobalt or iron) complexed to a polymeric material that contains phthalonitrile groups (e.g., preferably, poly(styrene-4-vinylphenoxyphthalonitrile)). Such an inventive polymer-metal complex may include a graft copolymer, with examples of a graft copolymer mentioned above. Preferred examples of a polymer-metal complex according to the invention are poly(styrene-4-vinylphenoxyphthalonitrile) complexed to cobalt; a polymer-metal complex having a saturation magnetization of at least 90 emu/g or greater; a polymer-metal complex that is carbon-encased, etc.

In an additional preferred embodiment, the invention provides at least one highly magnetic nanoparticle, wherein the nanoparticle is encased in carbon, and the encased nanoparticle is highly magnetic having a saturation magnetization of at least about 90 emu/g, such as, e.g., a nanoparticle comprising a derivative of a phthalonitrile composition and a Group VIII metal; etc.; a nanoparticle including poly(styrene-4-vinylphenoxyphthalonitrile) and cobalt; nanoparticles comprising a Group VIII metal (preferably cobalt); etc.

Another preferred embodiment of the invention provides a method of making a styrene polymer, comprising at least the steps of: (A) in a polystyrene compound containing a silyl ether bond, cleaving the silyl ether bond in a deprotection reaction to yield a deprotected polystyrene compound; (B) reacting the deprotected polystyrene compound with a phthalonitrile compound, to yield a polystyrene polymer, such as, for example, a method wherein poly(styrene-b-4-vinylphenoxyphthalonitrile) is made and the method includes: (A′) cleaving the silyl ether bond of poly(styrene-b-tert-butyldimethylsilyloxystyrene) to yield poly(styrene-b-4-vinylphenol); (B′) reacting the poly(styrene-b-4-vinylphenol) with 4-nitrophthalonitrile to yield poly(styrene-b-4-vinylphenoxyphthalonitrile).

In another preferred embodiment, the invention provides a method of making a Group VIII metal ferrofluid, comprising at least the step of: thermolysis of a reagent containing a Group VIII metal (with examples of reagents being dicobalt octacarbonyl, iron pentacarbonyl, etc.) in a phthalonitrile solution (such as a poly(styrene-b-4-vinylpheyxoyphthalonitrile) solution, etc.). The phthalonitrile solution may be concentrated, such as a concentrated poly(styrene-b-4-vinylpheyxoyphthalonitrile) solution. Such inventive methods of making a Group VIII metal ferrofluid optionally may include refluxing toluene in the presence of a poly(styrene-b-4-vinylphenoxyphthalonitrile) copolymer.

In another preferred embodiment, the invention provides a method of making a carbon-encased, stable, magnetic nanoparticle, comprising at least the step of: pyrolyzing Group VIII metal-nanoparticles (such as cobalt nanoparticles, etc.) to form carbon-encased nanoparticles. The Group VIII metal-nanoparticles may be stabilized before the pyrolyzing step; a pre-pyrolyzing step of stabilizing Group VIII metal-nanoparticles with copolymers (such as, e.g., poly(styrene-b-4-vinylphenoxyphthalonitrile) copolymers, etc.) may be included in the method of making a carbon-encased, stable, magnetic nanoparticle; and/or the method may include concentrating ferrofluids to a solid state and pyrolyzing the concentrated ferrofluids. An example of a preferable pyrolyzing temperature is a temperature of about 700° C.

The invention in a further preferred embodiment provides a method of making a graphitic-encased nanoparticle which has functional groups on its surface, comprising at least the steps of: pyrolyzing Group VIII metal-nanoparticles to form carbon-encased nanoparticles; functionalizing the carbon-encased nanoparticles with functional groups (such as, e.g., amine; isocyanate; hydroxyl; polymers (such as, e.g., polypeptides, polyglycolides, polylactides, poly(lactide-co-glycolides), poly(ethylene oxide), poly(ethylene oxide-co-propylene oxide), etc.); proteins (such as, e.g., streptavidin, avidin, etc.); polynucleotides; biospecific groups (such as, e.g., vitamins, steroids, etc.); etc.) to provide functional groups on a surface of the carbon-encased nanoparticles.

Another preferred embodiment of the invention provides a method of forming magnetic metal nanoparticles, comprising: preparing block copolymer dispersants for magnetic metals including at least one anchor block and at least one tail block, wherein the block copolymer comprises at least one block that is a precursor for a protective shell; followed by dispersing an organometallic metal precursor for a magnetic metal in a block copolymer solution and reacting with the block copolymer dispersants to form copolymer-magnetic metal nanoparticles; followed by heating the copolymer-magnetic metal nanoparticles to anneal the metal and form protective shells around the metal nanoparticles. In such inventive methods of forming magnetic metal nanoparticles, optionally, the magnetic metal nanoparticles may be protected with oxygen impermeable protective coatings; and/or optionally a step may be included of refunctionalization to provide dispersibility or biospecific reactivity in biological fluids.

### BRIEF SUMMARY OF THE DRAWINGS

FIG. 1A is a scheme for the synthesis of poly(styrene-b-tert-butyl-dimethylsilyloxystyrene).

FIG. 1B is a scheme for the inventive synthesis of poly(styrene-b-4-vinylphenoxyphthalonitrile), which is a novel composition of matter.

FIGS. 2A, 2B are transmission electron micrographs (TEM) of magnetic cobalt nanoparticles encapsulated by poly(styrene-b-4-vinylphenoxyphthalonitrile) copolymer.

FIGS. 3A, 3B are scanning electron micrographs (SEM) of a pyrolyzed cobalt-copolymer complex.

FIG. 4 is a hysteresis loop of cobalt nanoparticles stabilized with a 28,000 g/mole poly(styrene-b-4-vinylphenoxyphthalonitrile) copolymer after pyrolysis at 700° C. for 4 hours.

FIG. 5A includes DSC thermograms for poly(styrene-b-[tert-butyldimethylsilyl)oxy]-styrene, poly(styrene-b-phenol) and poly(styrene-b-4-vinylphenoxyphthalonitrile). FIG. 5B shows thermogravimetric analyses of poly(styrene-b-4-vinylphenoxyphthalonitrile).

FIGS. 6A, 6B are diagrams of behavior of cobalt magnetic fluids, in the absence (FIG. 6A) and presence (FIG. 6B) of an applied magnetic field.

FIG. 7 is a diagram of synthesis of inventive cobalt ferrofluids.

FIG. 8 is a diagram of hydrolytic decomposition of a microsphere. The high moment, oxidatively-stable nanoparticles can be incorporated into polymeric microspheres which can also incorporate other components such as therapeutic components, then the microsphere can be hydrolytically or enzymatically degraded in-vivo to release the therapeutic agents.

FIG. 9 is a scheme for pyrolyzing poly(styrene-b-4-vinylphenoxyphthalonitrile).

FIG. 10 is a graph of saturation magnetization of a cobalt-phthalonitrile complex at different elevated annealing temperatures.

FIG. 11 is a chart of saturation magnetization over time confirming oxidative stability for cobalt nanoparticles with a graphitic coating, a phthalonitrile network, an untreated phthalonitrile anchor block, and a triazine network.

FIG. 12A is the chemical structure of the compound subjected to NMR analysis as shown in FIG. 12B which is an NMR spectra analysis for poly(styrene-b-4-vinylphenoxyphthalonitrile).

FIG. 13 is an FT-IR analysis for poly(styrene-b-4-vinylphenoxyphthalonitrile).

FIG. 14 is an image of a dispersion of dicobalt octacarbonyl encased in a poly(styrene-b-vinylphenoxyphthalonitrile) block copolymer, cast from toluene onto a carbon paper coated TEM grid. The image confirms encapsulation of the cobalt precursor into aggregates encased within the phthalonitrile block copolymer.

### DETAILED DESCRIPTION OF A PREFERRED EMBODIMENT OF THE INVENTION

The present invention recognizes and exploits the advantages (such as greater magnetism) that may be achieved by forming and/or introducing phthalonitrile functional groups and a Group VIII metal (such as, e.g., cobalt, iron, etc.) into a composition of matter, preferably providing a magnetic fluid.

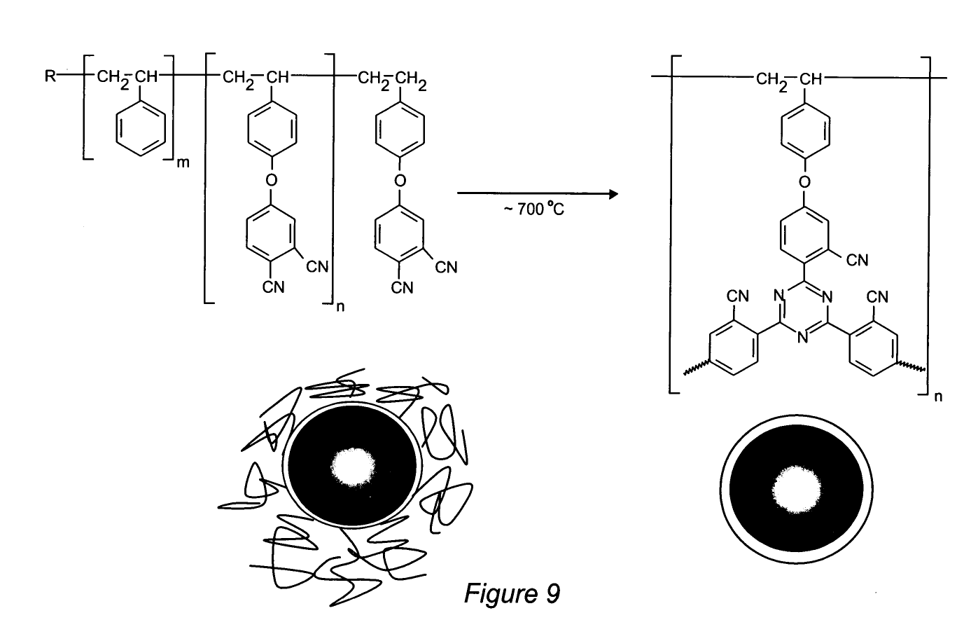

For example, by initially forming a magnetic fluid comprised of a metal copolymer complex according to the present invention, high concentrations of the magnetic metal (such as cobalt, iron, etc.) may be achieved. By pyrolysis of the metal copolymer complex, there may be provided a material that has an extremely high saturation magnetization (95-100 emu/g material). Desirable magnetic qualities and oxidative stability can be provided by these inventive materials. FIG. 9 shows a scheme for pyrolyzing group VIII metal nanoparticles encased in poly(styrene-b-4-vinylphenoxyphthalonitrile), which pyrolysis affords the metal nanoparticles encapsulated with protective carbonaceous coatings.

Examples of compositions that the invention provides, and uses, include: block or graft copolymers wherein at least one block contains phthalonitrile moieties; poly(styrene-b-vinylphenoxyphthalonitrile) block copolymers comprising a polystyrene block and a polyvinylphenoxyphthalonitrile anchor block and having the following structure (I): 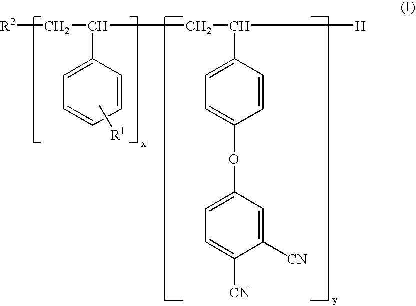
(in which x and y denote the average numbers of repeating units in the block copolymer and each can vary between about 10-500; R¹ can be —H or an alkyl substituent having about 1-100 carbon atoms, aromatic substituents (such as phenyl, biphenyl, naphthyl, benzophenone, or mixed alkyl aromatic substituents such as benzyl, phenylethynyl, ethynylbenzene, etc.); R² can be any alkyl or aromatic groups derived from an initiator (such as alkyl groups with 1-6 carbons or phenyl, etc.)); and, graft copolymers comprising a random sequenced mixed anchor block and PDMS tail grafts, with the structure of the graft copolymer being as follows in formula (II): 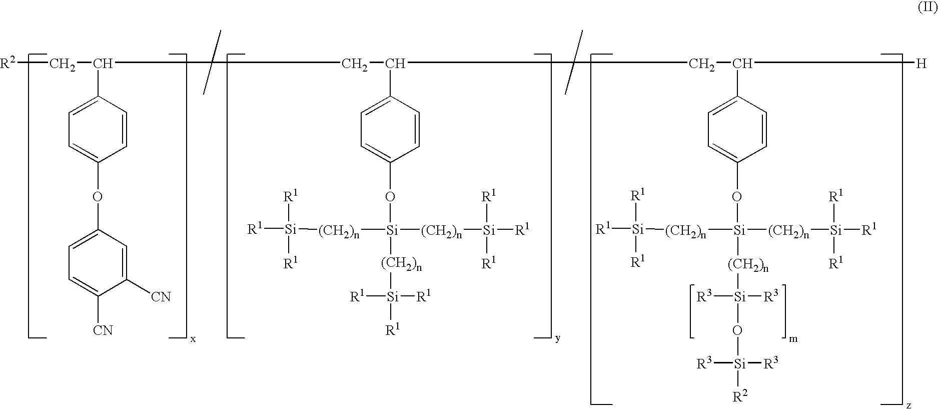
(wherein R¹ can be any group or combinations of groups that lead to a silica containing residue upon pyrolysis such as —OR; where R is any alkyl or aromatic group such as linear or branched alkyl groups containing about 1 to 6 carbons, aromatics such as phenyl, biphenyl, naphthyl, etc., or esters such as acetoxy; R² can be any alkyl or aryl group derived from an initiator moiety such as alkyl groups containing 1-6 carbons or phenyl; R³ can be alkyl groups containing about 1-6 carbons or aromatic groups such as phenyl, biphenyl, naphthyl, etc.; n is an integer of at least 1, preferably an integer from about 1-6; m preferably is an integer from about 10-500 and represents an average number of repeating units in the polysiloxane grafts; the relative compositions of the structural repeating units in the backbone of the graft copolymer denoted by x, y and z can vary substantially, preferably x, y and z can each vary from about 1 to 200 and they can be the same or different; with the lines across the backbone of the copolymer denoting that most embodiments of these copolymers contain the three types of repeating units in various statistical sequences).

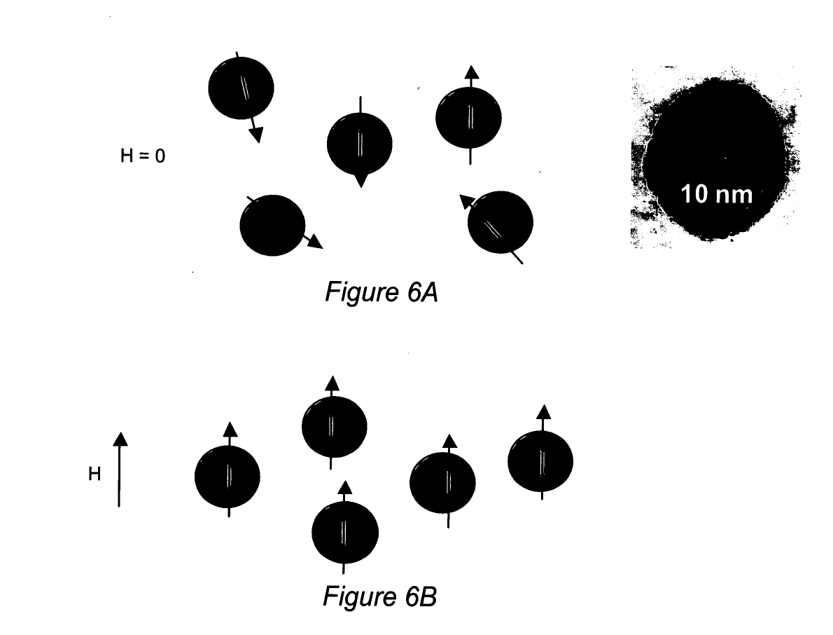

In the invention, as a group VIII metal for complexing, use of cobalt is particularly preferred. Cobalt nanoparticles behave as single domain particles each with their own magnetic dipole in the absence of an applied magnetic field (H). (FIG. 6A.) Cobalt nanoparticles exhibit net magnetization in the direction of an applied magnetic field and orient dipoles in a specific vector. (FIG. 6B.)

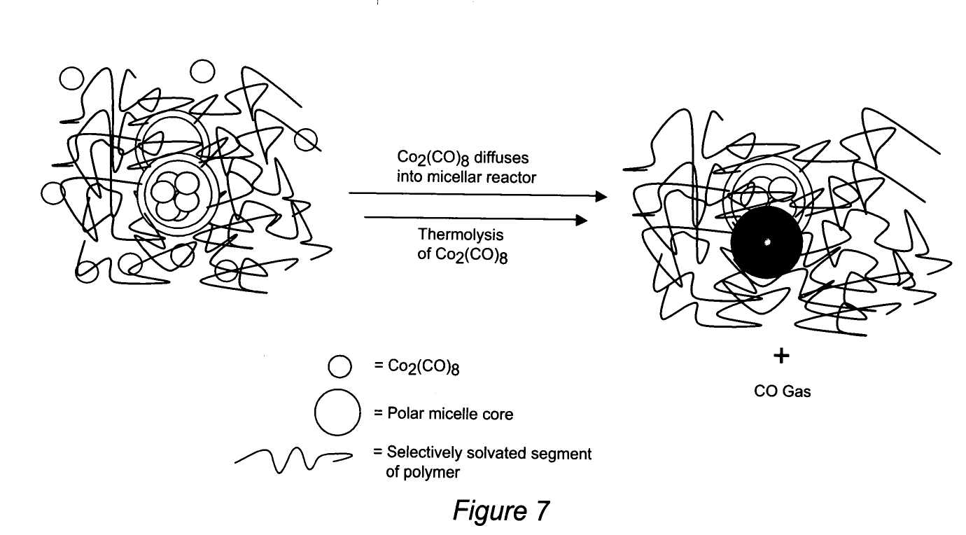

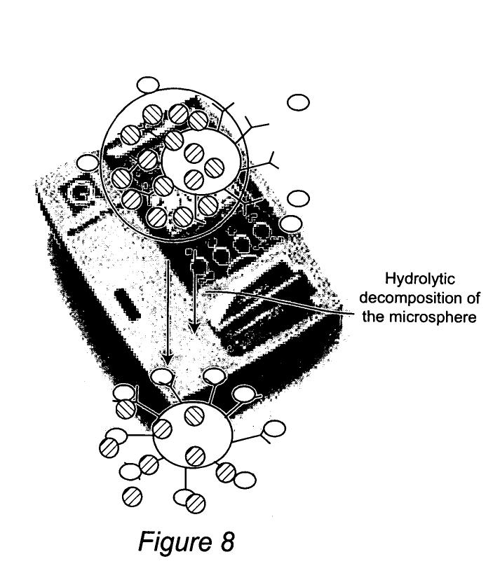

Cobalt ferrofluids may be synthesized via the thermolysis of CO₂(CO)₈ in solutions of macromolecular aggregates. (FIG. 7)

Uses of inventive nanoparticles and inventive ferrofluids (such as cobalt ferrofluids, cobalt nanoparticles, etc.) with high saturation magnetizations and oxidative stability include: targeted drug delivery applications, cell separations, separations of biomolecules, magnetic assay devices, quantitative measurements, magnetic removal of toxic materials from wastestreams; other biotechnological applications, etc.

Another use for inventive particles may be encapsulation within biodegradable microspheres along with a drug of interest to afford highly magnetic microspheres that may be localized to distinct regions within a human body. The present invention may be used in connection with hydrolytic and/or enzymatic decomposition of a microsphere. As shown in Fig., a microsphere may be subjected to hydrolytic decomposition.

Another use of materials according to the invention is to selectively isolate biomolecules, cells, and toxins from a variety of media by chemically modifying their surfaces. Materials according to the invention may be used in assays and waste water treatment.

For example, novel compositions, complexes, polymers, etc. according to the invention may be made as follows.

An example of a starting material to use is a block copolymer comprised of polystyrene and a polyvinylphenol block containing a silyl ether protecting group. The silyl ether bond may be cleaved in a deprotection reaction to yield a deprotected polystyrene compound, such as, e.g., cleaving the silyl ether bond of poly(styrene-b-tert-butyldimethylsilyloxystyrene) to yield poly(styrene-b-4-vinylphenol). The deprotected polystyrene compound then may be reacted with a phthalonitrile compound, such as, e.g., reacting poly(styrene-b-4-vinylphenol) with 4-nitrophthalonitrile. By reacting the deprotected polystyrene compound with the phthalonitrile compound, a polystyrene polymer may be produced (such as, e.g., poly(styrene-b-4-vinylphenoxyphthalonitrile, etc.).

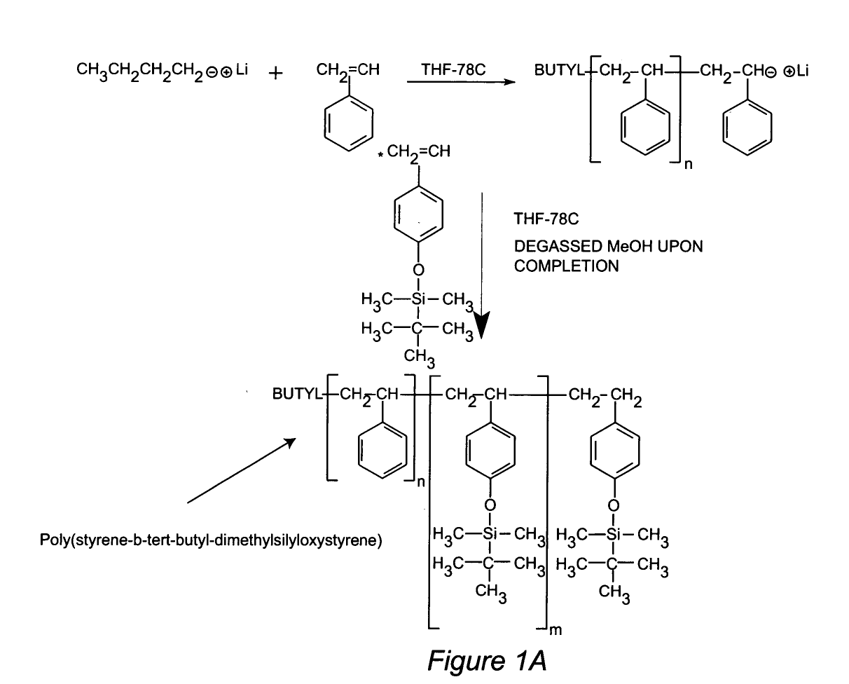

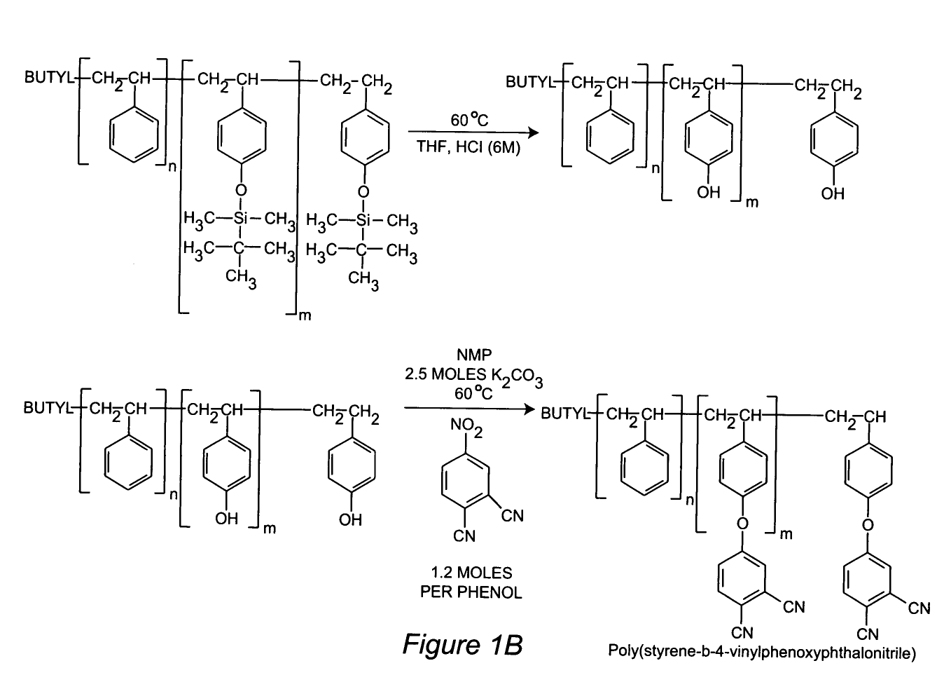

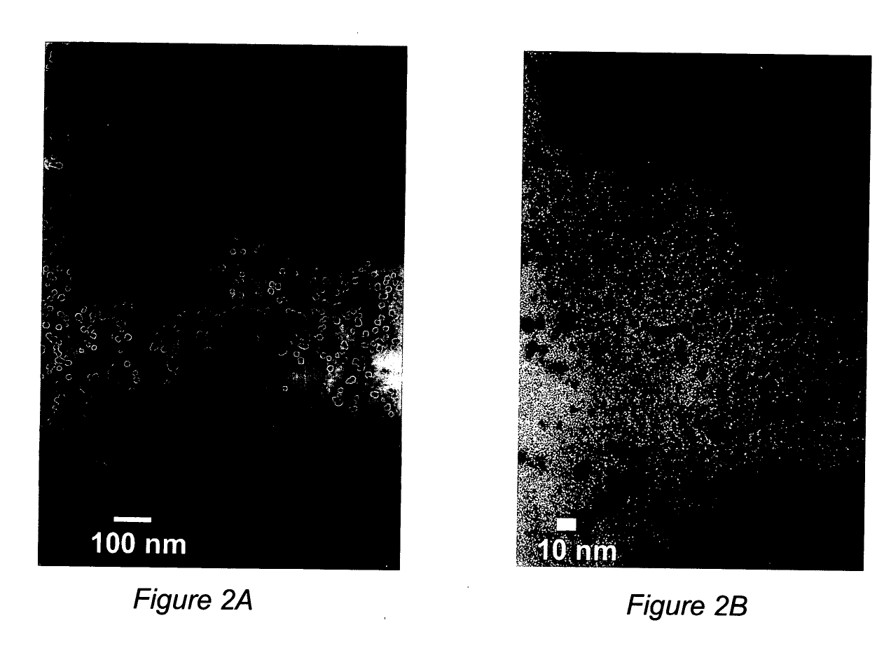

An example of a reaction scheme for making poly(styrene-b-tert-butyl-dimethylsilyloxy-tyrene is shown in FIG. 1A. The reagent, poly(styrene-b-tert-butyl-dimethylsilyloxystyrene, marked with an asterisk (*), is known, see, e.g., Hirao, A.; Koyoaki, K.; Takenaka, K.; and Nakahama, S.; Macromolecules 1993, 26, 4995-5003.

Block or graft copolymers containing phthalonitrile groups (such as, e.g., poly(styrene-b-4-vinylphenoxyphthalonitrile (for which a synthesis scheme is shown in FIG. 1B); etc.) may be used for making a copolymer solution containing phthalonitrile groups in which to conduct thermolysis of a reagent containing a Group VIII metal, thereby making a Group VIII metal ferrofluid. A preferred example of a group VIII metal to use is cobalt, and a preferred example of a reagent containing cobalt is dicobalt octacarbonyl. Alternatively, the group VIII metal can be iron, and a preferred reagent containing iron is iron pentacarbonyl. Preferably, the cobalt octacarbonyl and iron pentacarbonyl, respectively, are used in concentrated form. Preferably there is included a reflux step in the presence of the phthalonitrile solution, such as a step of refluxing toluene in the presence of the phthalonitrile solution.

For example, a cobalt ferrofluid may be formed in a poly(styrene-b-4-vinylphenoxyphthalonitrile) copolymer solution, such as by dissolving 0.50 g of block copolymer in toluene, followed by deoxygenating the solution (for 3 hours), followed by adding 1.0 g of CO₂(CO)₈, followed by refluxing at 110° C. (4 hours), to produce a weight ratio of copolymer:Co in the ferrofluid in a range of about 1.5-1.6 (or 1.5:1 to 1.6:1). This produces group VIII metal nanoparticles wherein each particle is encased in the block or graft copolymer containing the phthalonitrile groups.

A Group VIII metal ferrofluid may be further processed, such as to make carbon-encased, oxidatively stable, magnetic nanoparticles, such as by pyrolyzing Group VIII metal-nanoparticles (such as, e.g., cobalt nanoparticles, etc.) to form carbon-encased nanoparticles (such as, e.g., carbon-encased cobalt nanoparticles, etc.). The ferrofluids can be concentrated to a solid state, followed by treating the complexes at elevated temperatures. Preferably, pyrolyzing proceeds in a temperature range of about 500 to 700° C., with a pyrolyzing temperature of 700° C. mentioned as a preferred but non-limiting example.

Cobalt-graphitic complexes may be generated from the pyrolyzed complexes using grinding/filtering procedures, or using aggregated macromolecular solutions.

Carbon-encased nanoparticles may be further processed, to provide functional groups on a surface of the carbon-encased nanoparticles, such as by, e.g., an ammonia plasma treatment, etc. Thus, cobalt nanoparticles with surface functionality may be provided.

For example, copolymers may be synthesized with phthalonitrile pendant groups and ethoxy or methoxysilane to afford cobalt particles encapsulated with graphitic or silica protective coatings.

In the present invention, graft copolymers may be formed and used to form group VIII metal nanoparticle ferrofluids, such as cobalt nanoparticle ferrofluids, etc. Such complexes may be subjected to pyrolysis (such as to form a protective coating from the phthalonitrile graft copolymer and also to anneal the group VIII metal), followed by milling the resultant complexes, and functionalizing the complexes with amine groups. Preferably, such graft copolymers contain both a graphitic precursor (phthalonitrile) and a silica precursor (e.g., alkoxysilanes). The phthalonitrile portion produces a robust, oxygen-impermeable coating upon pyrolysis and the silica precursor provides a surface which can be functionalized with a range of functional groups. Graft copolymers according to formula (II) above may be mentioned, of which a preferred example of such a graft copolymer according to the invention is shown below in formula (II-A): 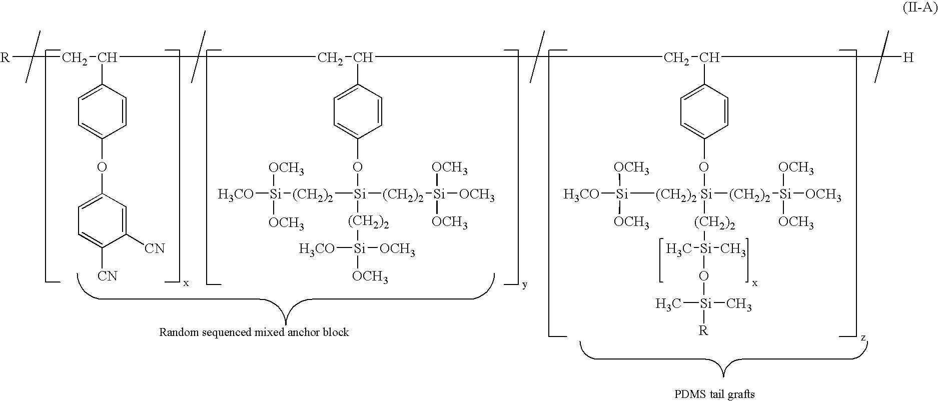
See also Examples, below, regarding graft copolymers according to the invention.

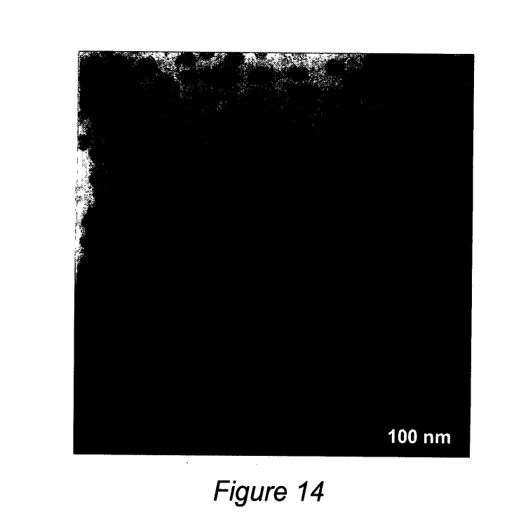

Also, block or graft copolymers can form ordered micellar solutions if dissolved in a solvent which is selective for one of the components (the so-called tail block). In designing the copolymer, the tail block should be selectively solvated by the solvent, while the anchor block should be relatively insoluble. The anchor block should coordinate with the organometallic precursor for the magnetic metal, so that solutions of the block (or graft) copolymer can form aggregates with the organometallic precursor within the block or graft copolymer solution structures (e.g., in the cores of micelles) (for an example, see the image of FIG. 14). The metal nanoparticles are then formed by conducting the reaction of the organometallic precursor to form the metal within the aggregate solution structures. The anchor block should form an oxygen impervious protective shell around the metal nanoparticles when the metal nanoparticle-copolymer complex is heated at an elevated temperature (e.g., 500-700° C.). In some cases, the heat treatments also anneal the metal into a particular crystal structure, and this results in higher magnetic moments. Because the copolymer forms a protective shell in the heat treatment, the metal nanoparticles are highly resistant to oxidation after the heat treatment. These heat-treated complexes are both very highly magnetic and also stable against oxidative damage (which would reduce the magnetization over time). The heat treatment partially pyrolyzes the organic copolymer during the process, so the resultant protective surface on the metal nanoparticles must be re-functionalized with biospecific groups or with polymeric dispersants to use these in biotechnological applications. Thus, dispersion of the oxidatively-stable, high-moment, magnetic nanoparticle complexes in water or biological fluids such as blood may be provided.

Another use for the present invention is that biocompatible, polydimethylsiloxane-based ferrofluids may be designed using the present invention, for use in therapies for treating retinal detachment disorders.

The following inventive Examples are mentioned, but it will be appreciated that the invention is not limited to the Examples.

### EXAMPLE 1

A block copolymer was synthesized through the sequential anionic polymerization of styrene and tert-butyldimethylsilyloxystyrene to afford poly(styrene-b-tert-butyldimethylsilyloxy-styrene). The silyl ether bonds of the copolymers were hydrolyzed under acidic conditions to afford poly(styrene-b-4-vinylphenol). The pendant phenols of the copolymer were chemically modified with 4-nitrophthalonitrile under basic conditions to afford poly(styrene-b-4-vinylphenoxyphthalonitrile). A stable suspension of metallic cobalt or iron nanoparticles were formed through the thermolysis of either dicobalt octacarbonyl or iron pentacarbonyl in concentrated solutions of toluene and poly(styrene-b-4-vinylphenoxyphthalonitrile). The nanoparticles were concentrated to a solid form and pyrolyzed in a tube furnace at 700° C. under an argon purge for four hours. Vibrating sample magnetometry indicates the nanoparticles are oxidatively stable and retain their high magnetizations (˜95-100 emu/g) for over a year. Thus, there were synthesized poly(styrene-b-4-vinylphenoxyphthalnitrile metal nanoparticle complexes that afford highly magnetic (95-100 emu/g) material, oxidatively stable nanoparticles.

### EXAMPLE 2

The invention provides a distinct advantage over conventional technology by affording magnetic nanoparticles prepared in copolymer solutions where the copolymers encase the metal nanoparticles. Upon pyrolysis, these material have high saturation magnetizations (at least 95-100 emu/g material) and oxidative stability under ambient conditions. Although these inventive nanoparticles are somewhat adhered together by the coatings after the pyrolysis step, much of the small nanoparticle size is retained. For the inventive materials of this example, the coasting are brittle and these material can be ground into nanoparticles which have diameters of about 200 nm. These inventive particles are sufficiently small to be coated with polymeric and/or biospecific groups, and used in biomedical applications. Testing of the inventive particles shows that their desirable magnetic properties are not lost after exposure to ambient conditions for over a year.

### EXAMPLE 3

Oxidatively stable 10-12 nm cobalt nanoparticles having high saturation magnetizations were formed via the pyrolysis of cobalt nanoparticles encapsulated with poly(styrene-b-4-vinylphenoxyphthalonitrile).

### EXAMPLE 4

Synthesis of poly(styrene-b-tert-butyldimethylsilyloxystyrene). All polymerizations were performed with THF freshly distilled from a still containing Na and benzophenone. Styrene was distilled from calcium hydride and injected into a 250 mL round bottom flask with THF (10% solids). The reaction solution was cooled to −78° C. and degassed under vacuum. The initiator n-butyllithium was injected into the reaction medium and after approximately 5 minutes, a solution of freshly distilled tert-butyldimethylsilyloxystyrene (synthesized using procedure of Nakahama et al) dissolved in THF (10% solids) was transferred slowly via cannula into the reaction medium using an argon purge. The solution was terminated with degassed methanol after approximately 10 minutes. The polymer was precipitated in a solution of methanol. A quantitative yield of the copolymer was achieved.

### EXAMPLE 5

Synthesis of poly(styrene-b-4-vinylphenol). Deprotection of the tert-butyldimethylsilyl group was accomplished by hydrolyzing the silyl ether bond under acidic conditions. The block copolymer poly(styrene-b-tert-butyldimethylsilyloxystyrene) was dissolved in a 0.6 M solution of aqueous HCl in THF (10% solids) and heated to 50° C. overnight. The solution was concentrated using a rotovap, and the polymer was precipitated into water. The isolated copolymer was redissolved in THF and reprecipitated into water after which it was further purified by reprecipitation from THF to hexane. The polymer was dried in a vacuum oven for 48 hours at a temperature of 1001C. The yield of poly(styrene-b-4-vinylphenol) was quantitative.

### EXAMPLE 6

Synthesis of poly(styrene-b-4-vinylphenoxyphthalonitrile). To a solution of 1-methyl-2-pyrrolidinone (NMP), poly(styrene-b-4-vinylphenol) was charged and dissolved with rigorous stirring (20% solids). Potassium carbonate was added to the reaction mixture for a molar ratio of phenol to base of 1:3. A 20% molar excess of 4-nitrophthalonitrile with respect to phenol was then charged to the reaction, and the solution was heated to 90° C. for approximately 12 hours. The solution was filtered to remove salts, and the copolymer was isolated via precipitation into a beaker of methanol. The copolymer was dried in a vacuum oven at 100° C. for 48 hours. The yield of copolymer was quantitative.

### EXAMPLE 7

Formation of oxidatively stable cobalt nanoparticles. Approximately 50 mL of toluene distilled over calcium hydride was injected into a 3 neck round bottom flask flame dried under an argon purge and equipped with a condenser and mechanical stirrer. Poly(styrene-b-4-vinylphenoxyphthalonitrile) (0.50 g) was added, and the reaction medium was deoxygenated with aragon for 2 hours. One gram of dicobalt octacarbonyl was then charged to the flask, and the solution was heated to 120° C. for 5 hours. The toluene was removed under vacuum and the polymer-cobalt complex was pyrolyzed in a tube furnace under an argon purge for 4 hours.

Results.

The block copolymer poly(styrene-b-tert-butyldimethylsilyloxystyrene) was synthesized through the sequential anionic polymerization of styrene and tert-butyldimethylsilyloxystyrene. Tetrahydrofuran was used as the reaction solvent since it readily solvates both monomers as well as high molecular weight poly(styrene-b-tert-butyldimethylsilyloxystyrene) copolymers. In order to avoid cleaving the silyl ether bond of tert-butyldimethylsilyloxystyrene, the anionic polymerizations were conducted at −78° C. Upon the addition of the n-butyllithium to the degassed solution of styrene and THF, the reaction medium turned dark red indicative of living polystyrl anions. The solution remained dark red after the addition of tert-butyldimethylsilyloxystyrene suggesting transfer and propagation of the living anionic species occurred after the addition of the second monomer.

Proton NMR was used to monitor the reaction by observing the disappearance of the vinyl protons of both monomers (δ8=5.2, 5.6, and 6.7 ppm). Proton NMR of an aliquot of the reaction medium immediately after the addition of n-butyllithium indicated quantitative conversion of the vinyl protons occurred immediately after the addition of the organolithium initiator.

In like fashion, ¹H NMR of the reaction medium immediately after the addition of tert-butyldimethylsilyloxystyrene also indicated quantitative conversion of the monomer occurred within seconds of its addition. The rapid reaction rates were commensurate with the fact that THF affords free ions and consequently rapid anionic polymerization kinetics.

A series of poly(styrene-b-tert-butyldimethylsilyloxystyrene) block copolymers were synthesized and converted to poly(styrene-b-4-vinylphenoxyphthalonitrile). (Table 1). The motivation for synthesizing copolymers with smaller compositions of tert-butyldimethylsiliyloxy-styrene relative to styrene is based on the observation that copolymers used to form stable cobalt ferrofluids need to be soluble in nonpolar organic solvents such as toluene or chlorobenzene in order to facilitate the formation of stable cobalt ferrofluids. Because the protected phenol blocks are ultimately converted to phthalonitrile anchor blocks that re not soluble in toluene or chlorobenzene, the molecular weight of the tert-butyldimethylsilyloxystyrene blocks were kept significantly lower than the polystyrene blocks which are readily solvated in toluene.

TABLE 1
Summary of examples of inventive
poly(styrene-b-tert-butyldimethylsilyloxystyrene copolymers)

| | Targeted | Targeted | |
|---|---|---|---|
| Polystyrene | Protected | Mₙ | |
| Block Mₙ | Phenol Block Mₙ | (GPC) | PDI |
| 20,000 | 5,000 | 28,000 | 1.03 |
| 30,000 | 5,000 | 35,900 | 1.01 |
| 40,000 | 10,000 | 51,600 | 1.01 |
| 50,000 | 10,000 | 58,400 | 1.06 |
| 75,000 | 10,000 | 80,000 | 1.08 |

### EXAMPLE 8

### Synthesis of poly(styrene-b-4-vinylphenol) via the deprotection of poly(styrene-b-tert-butyldimethylsilyloxystyrene)

The silyl ether bond of poly(styrene-b-tert-butyldimethylsilyloxystyrene) was readily cleaved in an acid promoted deprotection reaction. The bock copolymer was deprotected in THF (0.6 M HCl aqueous) to afford poly(styrene-b-4-vinylphenol). The polymer was precipitated into cold water and reprecipitated from THF to hexane. Proton NMR of the isolated block copolymer indicated quantitative deprotection of the tert-butyldimethylsilyloxystyrene block was achieved as the t-butyl and dimethyl proton sets of the protecting group at 1.0 and 0.2 ppm respectively were not present in the isolated copolymers after deprotection. Moreover, the hydroxyl functionality of poly(styrene-b-4-vinylphenol) was also observed using FT-IR as indicated by the presence of the hydroxyl stretch at approximately 3500 cm^{-1.}

### EXAMPLE 9

### Synthesis of poly(styrene-b-4-vinylphenoxyphthalonitrile) via the chemical modification of poly(styrene-b-4-vinylphenol) with 4-nitrophthalonitrile

Poly(styrene-b-4-vinylphenoxyphthalonitrile) was synthesized by chemically modifying poly(styrene-b-4-vinyl-phenol) with 4-nitrophthalonitrile under basic conditions in NMP. Proton NMR of the isolated copolymers indicated that the chemical modification reaction was quantitative. For a given copolymer, the ratio of the proton integrations of the proton set ortho to the phthalonitrile functionality to the aromatic protons of the styrene block were in agreement. For instance, for a poly(styrene-b-4-vinylphenoxyphthalonitrile) copolymer with a polystyrene block molecular weight of 20,000 g/mole and a 4-vinylphenoxyphthalonitrile block molecular weight of 5,255 g/mole, the theoretical ratio of the aromatic proton set ortho to the phthalonitrile functional groups to the remaining, overlapping aromatic proton sets is ˜22/1093. The experimental ratio of these protons determined using ¹H NMR was ˜22.4/1093 thus suggesting the pendant phenols of poly(styrene-b-4-vinylphenol) were quantitatively derivatized with 4-nitrophthalonitrile. Fourier transform infrared spectroscopy also confirmed the chemical modification of the pendant phenols with 4-nitrophthalonitrile as the characteristic stretch of nitrile moieties was observed in all copolymer at ˜2230 cm⁻¹.

### EXAMPLE 10

Cobalt ferrofluid formation. Cobalt ferrofluids were formed through the thermolysis of dicobalt octacarbonyl in concentrated solutions of poly(styrene-b-4-vinylpheyxoyphthalonitrile). The ferrofluids were formed by refluxing toluene at 110° C. for 5 hours in the presence of 0.50 g of poly(styrene-b-4-vinylphenoxyphthalonitrile) copolymers derived from the poly(styrene-b-tert-butyldimethylsilyloxystyrene) copolymers included in Table 1 above. Transmission electron microscopy indicates the copolymers were successfully used to form well-dispersed cobalt nanoparticles approximately 8-10 nm in diameter.

Vibrating sample magnetometry was used to evaluate the ferrofluids for superparamagnetic behavior. Hysteresis loops of the ferrofluids revealed superparamagnetic behavior as zero magnetization was observed when the applied magnetic field was removed. This indicates the ferrofluids lack remnant magnetization and randomly disperse after an applied magnetic field was removed.

### EXAMPLE 11

### Pyrolysis of cobalt nanoparticles encapsulated with poly(styrene-b-4-vinylphenoxyphthalonitrile)

Cobalt nanoparticles stabilized with poly(styrene-b-4-vinylphenoxyphthalonitrile) copolymers were pyrolyzed to form carbon encapsulated nanoparticles by concentrating the ferrofluids to a solid state and pyrolyzing the copolymer coated nanoparticles at 700° C. in a tube furnace under an argon purge. Scanning electron microscopy of the pyrolyzed materials reveals ˜10-15 nm particles. Vibrating sample magnetometry indicates the cobalt particles have minimal hysteresis and high saturation magnetization values (˜95-100 emu/g). The high saturation magnetizations of the cobalt nanoparticles were retained for over 200 days under ambient conditions.

The above experiments and Examples show that poly(styrene-b-4-vinylphenoxyphthalonitrile) may be used to form stable dispersions of magnetic cobalt nanoparticles ˜10 nm in diameter. These particles may be converted to oxidatively stable entities by pyrolysis at 700° C. After pyrolysis of the polymer-cobalt complex, the cobalt nanoparticles maintain their spherical morphology, have minimal hysteresis, and retain their high saturation magnetizations (˜95-100 emu/g) under ambient conditions.

### EXAMPLE 12

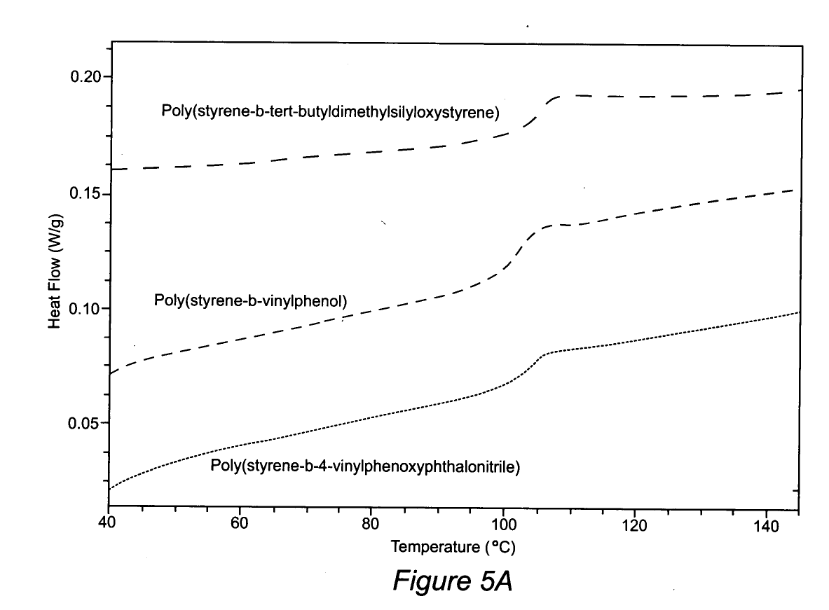

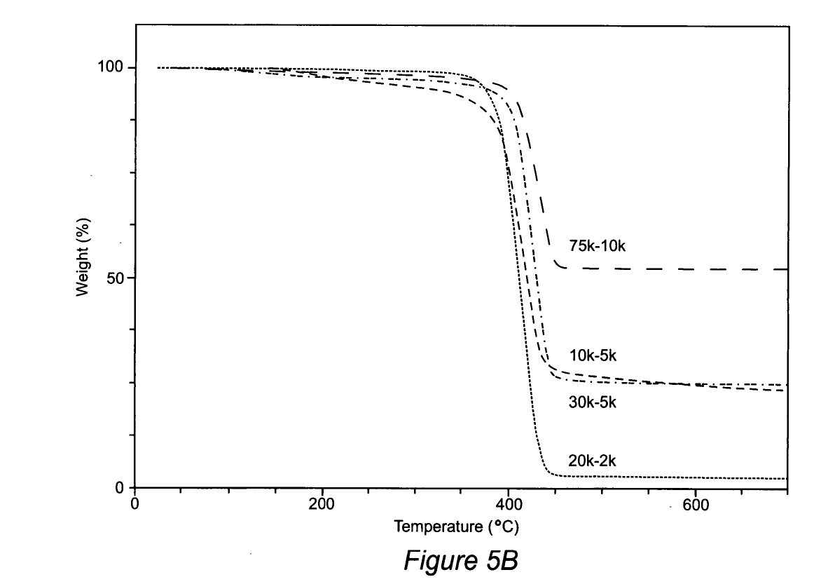

Universal V3.3B TA Instruments were used, and DSC thermograms were prepared for a 50k-20k diblock series, with thermograms for poly(styrene-b-[tert-butyldimethylsilyl)oxy]-styrene, poly(styrene-b-phenol) and poly(styrene-b-4-vinylphenoxyphthalonitrile). (FIG. 5A.) FIG. 5A suggests that phase separation does not occur with starting or chemically modified block copolymers.

Universal V3.3B TA Instruments were used, and thermogravimetric analysis of poly(styrene-b-4-vinylphenoxyphthalonitrile) was performed, at a ramp rate of 10° C./minute, in a nitrogen atmosphere, with the results shown in FIG. 5B.

### EXAMPLE 13

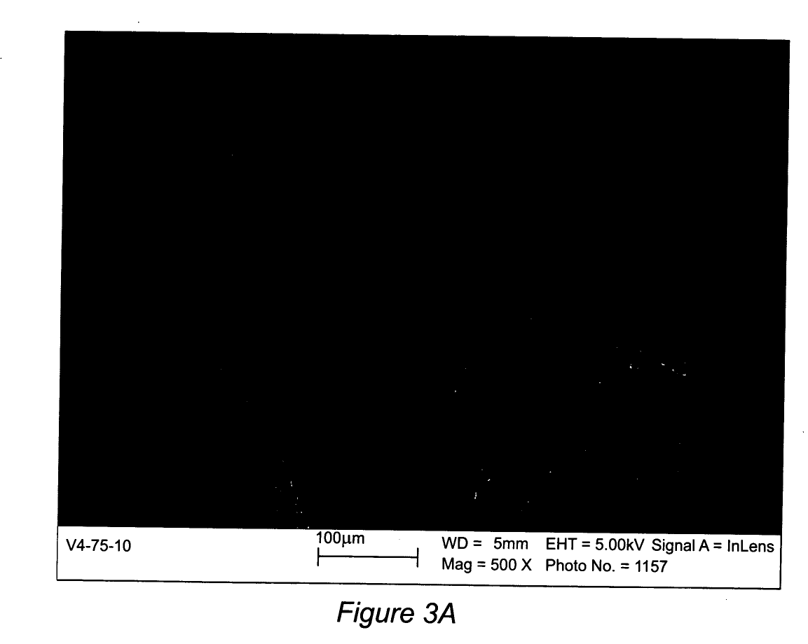

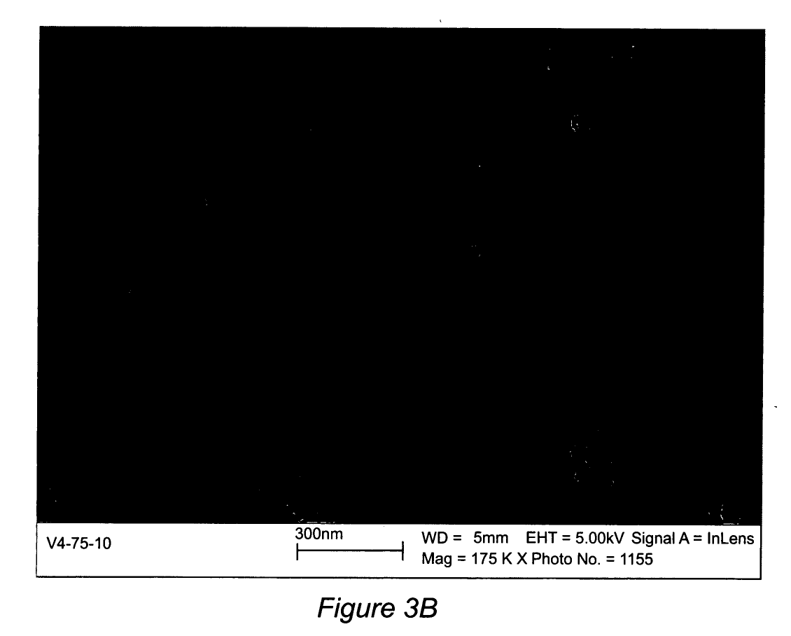

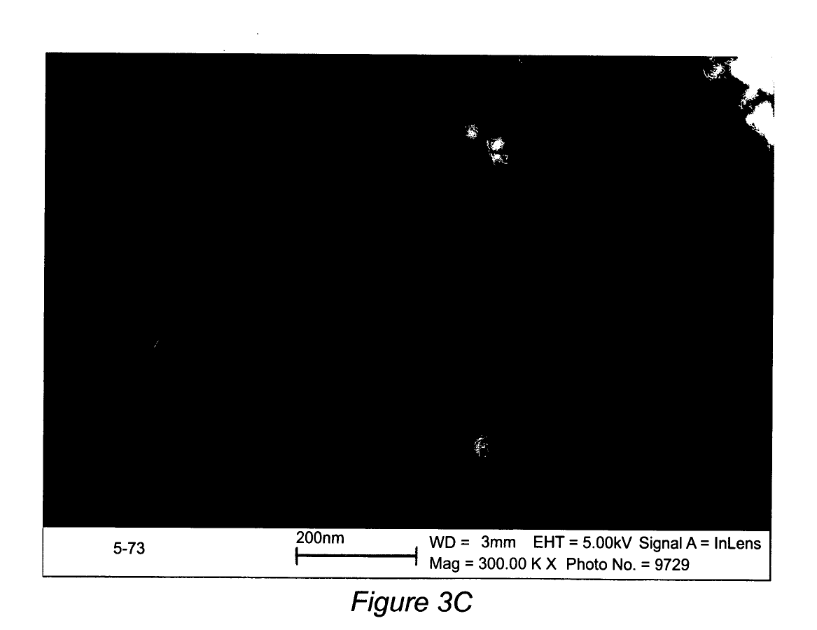

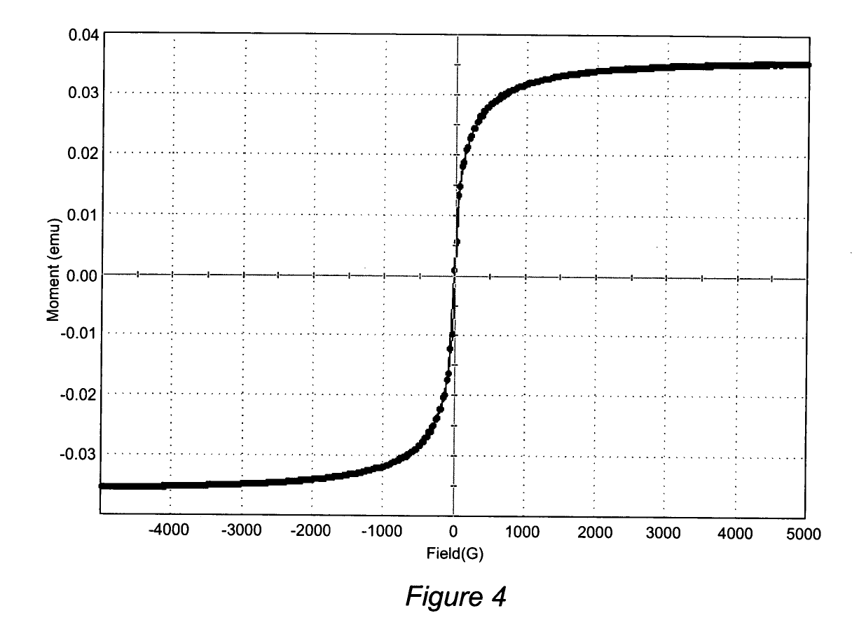

An example of a procedure for charring cobalt particles coated with poly(styrene-b-4-vinylphenoxyphthalonitrile) is as follows: remove toluene under vacuum, followed by pyrolysis at 700° C. for 4 hours under argon, to produce a pyrolyzed cobalt-copolymer complex. FIGS. 3A, 3B are SEM micrographs of a pyrolyzed cobalt-copolymer complex. In the SEM micrograph in FIG. 3B, formation of a particulate film is revealed for the subject pyrolyzed cobalt-copolymer complex.

Pyrolyzed cobalt particles were ground for two hours in a planetary ball mill grinder at 700 RPM with 10 M KOH/H₂O, after which SEM images were taken. (FIG. 3C.)

### EXAMPLE 14

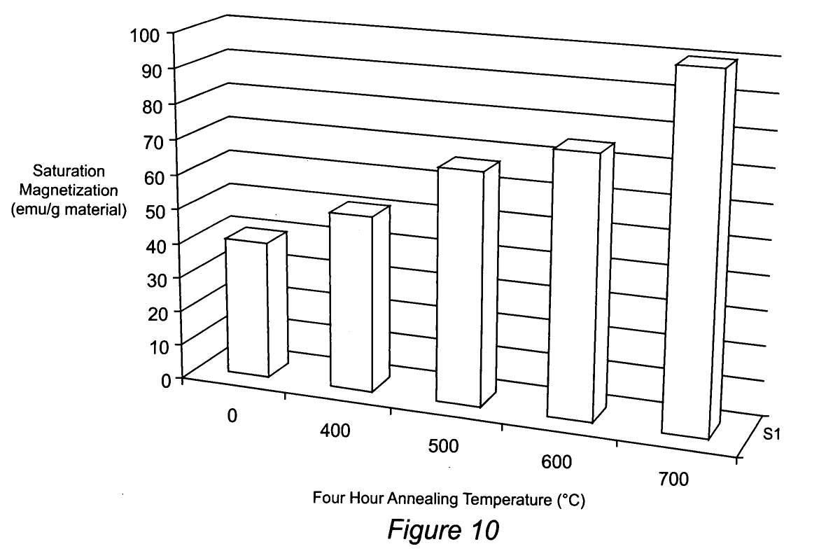

A cobalt-phthalonitrile complex was annealed at different elevated temperatures, for four hours, at 400, 500, 600 and 700° C., respectively. The results are shown in FIG. 10, from which it can be seen that annealing cobalt-phthalonitrile complexes at elevated temperatures increases saturation magnetization.

### EXAMPLE 15

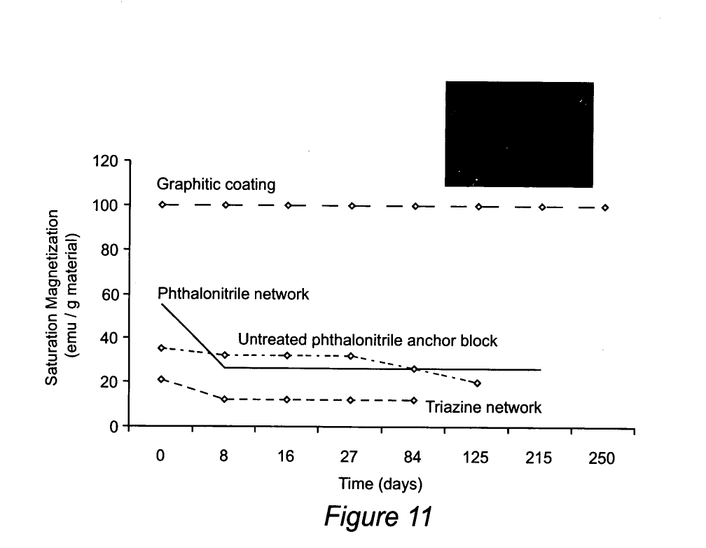

Oxidative stability under ambient conditions was measured for each of a graphitic coating, a phthalonitrile network, an untreated phthalonitrile anchor block and a triazine network. Cobalt particles coated with pyrolyzed poly(styrene-b-4-vinylphenoxyphthalonitrile) were used. The results are shown in FIG. 11, from which it can be seen that cobalt particles coated with pyrolyzed poly(styrene-b-4-vinylphenoxyphthalonitrile) are oxidatively stable under ambient conditions.

Thus, poly(styrene-b-4-vinylphenoxyphthalonitrile) may be synthesized by the sequential anionic polymerization of styrene and tert-butyldimethylsilyloxystyrene followed by the chemical modification of the template block copolymer. TGA analysis of the poly(styrene-b-4-vinylphenoxyphthalonitrile) copolymers indicates encouraging char yields may be achieved during pyrolysis. Superparamagnetic cobalt ferrofluids may be generated from the thermal decomposition of CO₂(CO)₈ in toluene solutions containing poly(styrene-b-4-vinylphenoxyphthalonitrile). Pyrolyzing the cobalt-copolymer complexes at 700° C. affords highly magnetic (100 emu/g material), oxidatively stable cobalt nanoparticles.

### EXAMPLE 16

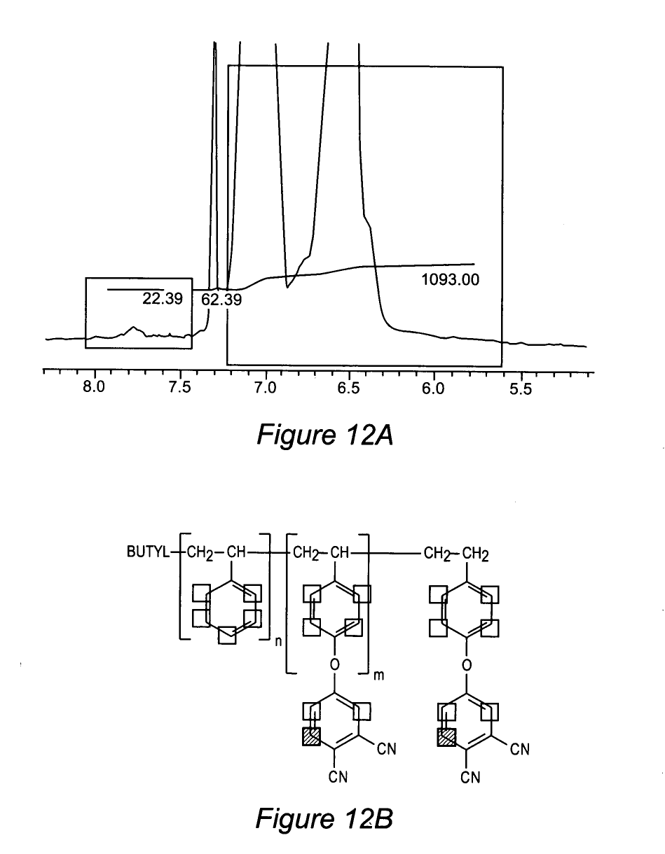

¹H NMR spectra measurements were made for the compound shown in FIG. 12B. ¹H NMR (FIG. 12A) indicates quantitative chemical modification of 4-vinylphenol block with 4-nitrophthalonitrile. Theoretical ratio of resolved phthalonitrile proton set to overlapping aromatic proton sets: 22/1094. Observed ratio of protons: 22.4/1093.

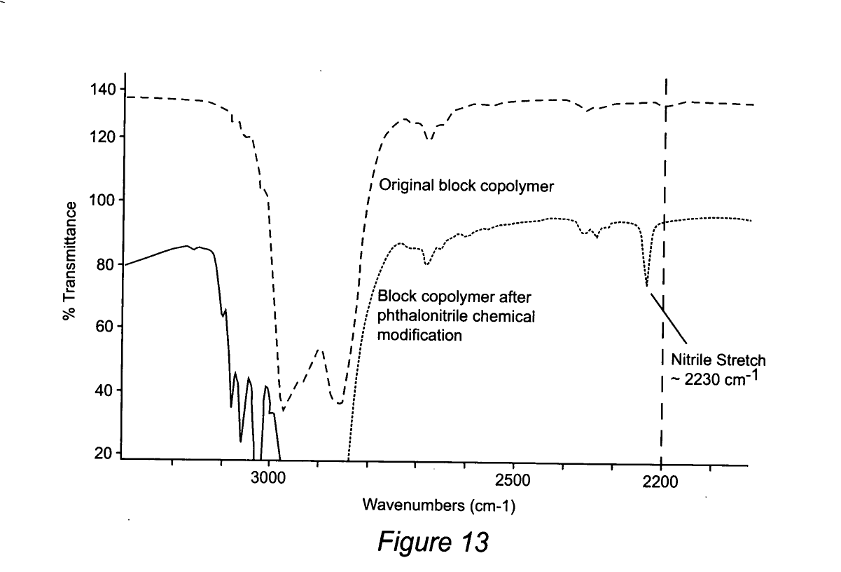

FT-IR analysis was performed for poly(styrene-b-4-vinylphenoxyphthalonitrile), and the results are shown in FIG. 13. Phthalonitrile functionality in the block copolymer may be seen.

### EXAMPLE 17

Synthesis of poly(4-acetoxystyrene). 4-Acetoxystyrene (4 g, 25 mmol) was charged to a 50 mL single-neck, round-bottom flask containing 10 mL of chlorobenzene. Benzoyl peroxide (60 mg, 0.25 mmol) was added, and the reaction medium was deoxygenated using an argon purge for 3 hours. The reaction was subsequently heated at 90° C. for 12 h. The polymer was precipitated by pouring the reaction solution into a beaker of stirring hexane. The polymer was filtered and dried under vacuum at 100° C. for 24 h. The yield was approximately 75% (3 g).

Synthesis of poly(4-vinylphenol). Poly(4-acetoxystyrene) was deacetylated to afford poly(4-vinylphenol) by charging 2 g of the polymer to a 50 ml, 2-neck, round-bottom flask equipped with a magnetic stirrer and a reflux condenser. Approximately 20 mL of a 15 M solution of NH₄OH in methanol was injected, and the slurry was heated to methanol reflux (65° C.) for 12 h. The medium became transparent, and the polymer was isolated by pouring the reaction solution into a beaker of stirring water. The polymer was filtered and dried at 150° C. for 24 h. Approximately 90% yield was obtained (1.8 g).

Synthesis of poly(4-vinylphenol-co-4-vinylphenoxyphthalonitrile). An exemplary procedure for the synthesis of a poly(4-vinylphenol-co-4-vinylphenoxyphthalonitrile) copolymer with an equivalent number of phenol and phthalonitrile repeat units is provided. 1 g of a 16,680 g/mole poly(4-vinylphenol) polymer (6.2 mmol phenol) was charged to a 50 ml single-neck, round-bottom flask equipped with a magnetic stirrer and septum seal. Anhydrous potassium carbonate (0.86 g, 6.2 mmol), 4-nitrophthalonitrile (0.54 g, 3.1 mmol) and 10 mL of NMP were added, and the reaction medium was heated at 90° C. for 12 h. The copolymer was isolated by precipitation in cold water, filtered, and dried at 75° C. under vacuum for 24 h. Approximately 85% yield was obtained.

Synthesis of Poly(4-vinylphenoxyphthalonitrile-co-4-vinylphenoxytrivinylsilane) 1 g of a 25,437 g/mole poly(4-vinylphenol-co-4-vinylphenoxyphthalonitrile) copolymer (2.7 mmol phenol) was charged to a 50-mL, single-neck, round-bottom flask equipped with a magnetic stirrer and septum seal. Approximately 10 mL of THF distilled over sodium was injected along with 1.01 mL of triethylamine (7.3 mmol) distilled from calcium hydride. The reaction solution was cooled to 0° C. and trivinylchlorosilane (0.51 mL, 3.3 mmol) was added dropwise with rigorous stirring. The reaction was allowed to slowly warm to room temperature and progress for 12 h. The solution was filtered to remove salts, and the copolymer was isolated via precipitation into a beaker of stirring hexane. The copolymer was filtered and dried under vacuum at 75° C. for 24 h. Approximately 80% yield was achieved.

Synthesis of Poly(4-vinylphenoxyphthalonitrile-co-4-vinylphenoxytriethoxysilane-g-dimethylsiloxane). A 32,943 g/mol poly(4-vinylphenoxyphthalonitrile-co-4-vinylphenoxytrivinylsilane) copolymer (1 g, 6.33 mmol vinyl) was added to a 50-mL, single-neck, round-bottom flask equipped with a magnetic stirrer, 10 mL of freshly distilled THF, and a septum seal. A 10,000 g/mol poly(dimethylsiloxane) functionalized with terminal hydrido groups on one end (3.16 g, 0.32 mmol) was injected along with 0.5 wt % of Karstedt's catalyst (Pt). The reaction medium was heated to 60° C. and after 12 h 1.12 mL (6.01 mmol) of triethoxysilane was injected. The reaction was maintained at 60° C. for an additional 12 h to hydrosilylate the remaining vinyl groups. The solvent was removed under vacuum at 100° C. for 24 h, and the yield was quantitative.

Synthesis of cobalt nanoparticles encased in poly(4-vinylphenoxyphthalonitrile-co-4-vinylphenoxytriethoxysilane-g-dimethylsiloxane). A 3-neck, 250-mL, round-bottom flask equipped with a condenser, mechanical stirrer, and a septum-capped neck was flame-dried under argon. Approximately 50 mL of toluene was injected into the flask. A poly(4-vinylphenoxyphthalonitrile-co-4-vinylphenoxytriethoxysilane-g-dimethylsiloxane) copolymer (1.0 g, 6.04×10⁻⁶ mole, 0.42 meq phthalonitrile) was added quickly and dissolved. The reaction medium was deoxygenated by purging argon through the solution for 2 h. One gram of dicobalt octacarbonyl (2.93 mmol) was quickly charged to the flask, and the reaction was refluxed at 110° C. for 5 h. The solution was cooled to room temperature, transferred to a flame-dried, septum-capped, 100-mL, round bottom flask via cannula, and stored under an argon atmosphere.

Pyrolysis of cobalt-graft copolymer complexes. Toluene was removed from the ferrofluid under vacuum at 100° C. for 24 h. Approximately 1 g of the cobalt-copolymer complex was placed in a ceramic boat positioned in the center of a quartz tube within a tube furnace. The material was heated under an argon purge at 700° C. for 4 h and cooled to room temperature.

Grinding of pyrolyzed cobalt-graft copolymer complexes. Approximately 0.25 g of the pyrolyzed cobalt-poly(4-vinylphenoxyphthalonitrile-co-4-vinylphenoxytriethoxysilane-g-dimethylsiloxane) complex was charged to a steel chamber within a planetary ball-mill grinder equipped with 7 steel balls. 5 mL of toluene was added, and the material was ground at 700 rpm for 2 h. The cobalt-pyrolyzed graft copolymer complex was poured into a 50-mL, single-neck, round-bottom flask and dried under vacuum at 150° C. for 24 h.

Functionalization of the surfaces of pyrolyzed cobalt-pyrolyzed graft copolymer particles. Approximately 0.25 g of milled cobalt particles encapsulated with pyrolyzed poly(4-vinylphenoxy-phthalonitrile-co-4-vinylphenoxytriethoxysilane-g-dimethylsiloxane) was charged to a flame-dried, 100-mL, 3-neck, round-bottom flask equipped with a mechanical stirrer, reflux condenser, and septum seal. Toluene (25 mL) and 5 mL of 3-aminopropyltrimethoxysilane (28.2 mmol) were injected, and the reaction slurry was heated at toluene reflux for 12 h with aggressive stirring. The cobalt particles were drawn to the bottom of the round-bottom flask using a magnet, and the reaction solution was decanted. Approximately 50 mL of chloroform was injected, and the cobalt slurry was stirred for 5 minutes. The particles were drawn to the bottom of the flask, and the chloroform was decanted. This washing procedure was repeated 3 times to remove any unbound 3-aminopropyl functional silane. The particles were dried under vacuum at 150° C. for 24 h.

### EXAMPLE 18

Poly(styrene-b-vinylphenoxyphthalonitrile) diblock copolymer templates were designed and prepared in living anionic polymerizations, so that the block lengths are well controlled. Toluene solutions of these copolymers and dicobalt octacarbonyl have been used to generate discreet cobalt nanoparticles. The polystyrene block of the copolymers is solvated well by toluene (the dispersion solvent), whereas the polyvinylphenoxyphthalonitrile block is only sparingly soluble in toluene. The nitrites apparently coordinate with dicobalt octacarbonyl so that the polyvinylphenoxyphthalonitrile serves as the so-called anchor block, and the polystyrene tail blocks protrude out into the solvent to form a sterically stabilizing corona for the complexes in the dispersions.

Synthesis of Diblock Copolymer Dispersion Stabilizers and Formation of Cobalt Nanoparticle Ferrofluids

Poly(styrene-b-tert-butyldimethylsilyloxystyrene) copolymers were synthesized in living anionic polymerizations via sequential polymerization of styrene, then tert-butyldimethylsilyl-oxystyrene as described by Nakahama et al. Tetrahydrofuran was employed as the reaction solvent since it readily solvates both monomers as well as high molecular weight poly(styrene-b-tert-butyldimethylsilyloxystyrene) copolymers. In order to avoid cleaving the silyl ether bond of tert-butyldimethylsilyloxystyrene during synthesis, the anionic polymerizations were conducted at −78° C. Upon adding the n-butyllithium initiator, the reaction medium turned dark red signifying the presence of living polystyryl anions. The solution remained dark red after adding the tert-butyldimethylsilyloxystyrene, again indicating the presence of active styrenic species.

Proton NMR was used to monitor the progress of the copolymerizations by observing the disappearance of the vinyl protons of both monomers (δ=5.2, 5.6, and 6.7 ppm). An aliquot of the reaction medium taken immediately after adding the n-butyllithium indicated that quantitative conversion of the vinyl protons had already occurred. Similarly, ¹H NMR of the reaction just after adding tert-butyldimethylsilyloxystyrene also indicated that quantitative conversion of the monomer occurred within seconds. The rapid reaction rates were anticipated since THF affords solvent-separated ion pairs and highly reactive active chain centers. A series of poly(styrene-b-tert-butyldimethylsilyloxystyrene) copolymers with systematically varied molecular weights was synthesized (Table 2).

TABLE 2
Molecular weights of poly(styrene-b-tert-butyldimethylsilyloxystyrene)
copolymers

| | Targeted | | |
|---|---|---|---|
| Targeted | Protected | | |
| Styrene Block | Phenol Block | Mₙ | |
| Mₙ | Mₙ | (GPC) | PDI |
| 20,000 | 5,000 | 25,000 | 1.05 |
| 30,000 | 5,000 | 35,900 | <1.03 |
| 40,000 | 10,000 | 51,600 | <1.03 |
| 50,000 | 20,000 | 80,000 | 1.04 |
| 50,000 | 10,000 | 58,400 | 1.06 |
| 75,000 | 10,000 | 80,000 | 1.08 |

Gel permeation chromatography of the copolymers revealed narrow, monomodal peaks with polydispersities reflective of living anionic polymerizations. In addition, ¹H NMR of the isolated copolymers revealed proton resonances indicative of the polystyrene and poly(tert-butyldimethylsilyloxystyrene) blocks.

The silyl ether bonds of poly(styrene-b-tert-butyldimethylsilyloxystyrene) were readily cleaved in an acid-promoted deprotection reaction in THF. Proton NMR of the block copolymers after acidic treatment indicated that quantitative deprotection of the poly(tert-butyldimethylsilyloxystyrene) block was achieved. The t-butyl and dimethyl proton sets at 1.0 and 0.2 ppm were absent from the spectra of the isolated copolymers. Moreover, the hydroxyl group of poly(styrene-b-4-vinylphenol) was observed in the FT-IR spectra of the deprotected copolymers at ˜3500 cm⁻¹.

Poly(styrene-b-4-vinylphenoxyphthalonitrile) was synthesized by chemically modifying poly(styrene-b-4-vinylphenol) with 4-nitrophthalonitrile via a nucleophilic aromatic substitution reaction under basic conditions in NMP (1-methyl-2-pyrrolidinone): 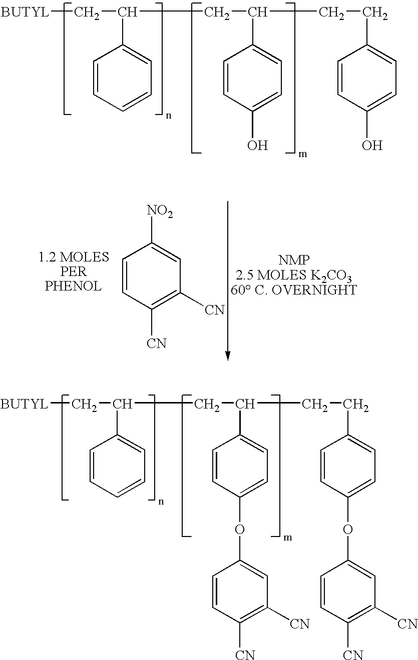
whereby poly(styrene-b-4-vinylphenoxyphthalonitrile was synthesized.

Proton NMR of the isolated copolymers indicated that the chemical modification reaction was quantitative. The integral ratios of the peaks due to the aromatic protons ortho to the phthalonitrile groups at 7.75 ppm to the remaining, overlapping aromatic proton resonances at 6.3-7.2 ppm were equivalent to the theoretical ratios for all of the phthalonitrile containing block copolymers. FT-IR also qualitatively confirmed chemical modification with 4-nitrophthalonitrile. The characteristic stretch of the nitrile moieties was observed in the poly(styrene-b-4-vinylphenoxyphthalonitrile) copolymers at ˜2230 cm⁻¹.

Dynamic weight loss profiles of a series of the diblock copolymers were evaluated by heating them in a TGA at temperatures up to 700° C. under a nitrogen atmosphere to determine whether the phthalonitrile groups in these materials led to significant residual carbonaceous char. An abrupt weight loss was observed for all of the diblock copolymers at ˜400° C., and the residue after that drop was retained by these materials up to the end of the experiments (700° C.). A copolymer with a 30,000 g mol⁻¹ polystyrene block and a 5,256 g mol⁻¹ polyvinylphenoxyphthalonitrile block retained about 20 wt % of carbonaceous residue, whereas a copolymer with a 75,000 g mol⁻¹ polystyrene block and a 10,512 g mol⁻¹ polyvinylphenoxyphthalonitrile block retained approximately 50% of its original weight. Such high residues after the elevated heat treatments were encouraging because it was reasoned that these residues might result in robust carbonaceous coatings for the cobalt nanoparticles.

A block copolymer with a 5,256 g mol⁻¹ phthalonitrile block molecular weight was used for investigating the formation of protective carbonaceous coatings around the cobalt nanoparticles. (It should be noted that all poly(styrene-b-4-vinylphenoxyphthalonitrile) copolymers derived from the template copolymers listed in Table 2 were successfully used to form stable suspensions of cobalt nanoparticles.) Cobalt ferrofluids were prepared by thermally decomposing dicobalt octacarbonyl in concentrated toluene solutions of poly(styrene-b-4-vinylphenoxyphthalonitrile). Fourier transform infrared spectroscopy was used to monitor the thermolysis of CO₂(CO)₈ and subsequent formation of metallic cobalt. FT-IR of the reaction mixture immediately after adding the CO₂(CO)₈ revealed carbonyl stretches at 2030, 2065, and 1858 cm⁻¹. Aliquots of the reaction medium after heating to 110° C. revealed the characteristic absorption bands of CO₄(CO)₁₂ at 2030, 2065, and 1858 cm⁻¹ as reported previously by Tannenbaum et al. After refluxing the reaction solution at 110° C. for five hours, the characteristic carbonyl stretches of CO₄(CO)₁₂ were absent.

Aliquots of the reaction solutions or dispersions were solvent-cast onto carbon paper on TEM grids to observe the nature of the aggregates during synthesis. Immediately after adding dicobalt octacarbonyl into the copolymer solution, cobalt aggregates as well as small cobalt species which may have been free in solution at this point in the reaction were visible. Soon after reaching the toluene reflux temperature, uniform cobalt aggregates were observed, possibly encased in micelles of the block copolymer. Transmission electron microscopy of solvent cast ferrofluids after four hours of reaction indicated that the precursor aggregates had densified and become smaller. Well-dispersed, spherical cobalt nanoparticles ˜8-10 nm in diameter had formed, probably encased in copolymer sheaths. It was thus demonstrated that the cobalt particles do form within the small copolymer aggregates. This probably also accounts for the relatively narrow particle size distribution, and the fact that each cobalt nanoparticle seems to be discreet.

Vibrating sample magnetometry was used to evaluate the magnetic properties of the ferrofluids. Magnetic hysteresis loops acquired at 25° C. revealed superparamagnetic behavior, characterized by zero magnetization when the applied magnetic field was removed. This suggests that the magnetic moments of the particles in these ferrofluids randomly disperse when the applied magnetic field is removed. Vibrating sample magnetometry of a representative copolymer-cobalt complex after solvent removal indicated that the complex had a specific saturation magnetization of ˜35 emu g⁻¹ material, but this had decreased to 20 emu g⁻¹ material after 125 days of exposure to ambient conditions. The decrease in saturation magnetization was attributed to the formation of antiferromagnetic cobalt oxide.

Magnetic susceptometry measurements (using a SQUID based sensor) were conducted on a dried pre-pyrolyzed sample. Room temperature measurements indicated a saturation magnetization of 30 emu g⁻¹ material (80 emu g⁻¹ Co), which is consistent with VSM measurements. However, a magnetic remanence of 7 emu g⁻¹ material (19 emu g⁻¹ Co) and a coercivity of 410 Oe were observed indicating that at least part of the sample is magnetically blocked at room temperature. Low temperature σ vs. H measurements indicated an asymmetric shift in the field-cooled hysteresis loop with respect to the zero field-cooled hysteresis loop. This loop shift is indicative of the coupling of an antiferromagnetic cobalt oxide layer with a ferromagnetic cobalt core (Nogues, J. and Schuller, I. K. Journal of Magnetism and Magnetic Materials. 1999; 192: 203-232.). This evidence supports the explanation for the decline in saturation magnetization over time. In addition, the cobalt specific magnetization (σ) showed a continuous increase with field at high fields and low temperature. However, at room temperature a almost saturated at high fields. These observations are consistent with the presence of paramagnetic species within the sample. This paramagnetic component is possibly unreacted cobalt carbonyl that has not been incorporated into the cobalt nano-crystals.

Preparation and characterization of cobalt nanoparticles encased in protective graphitic-like coatings

The complexes of cobalt metal nanoparticles coated with poly(styrene-b-4-vinylphenoxyphthalonitrile) were heated at elevated temperatures in efforts to convert the copolymers to carbonaceous coatings for protecting the metal against oxidation. The ferrofluid solvent was removed under vacuum, then the complexes were pyrolyzed at 400-700° C. in a tube furnace under an argon purge. This produced ensembles of particles adhered together by the pyrolyzed carbonaceous matrix. As expected based on the weight loss profiles of the copolymers (from TGA), some weight was lost in these processes. Thus, it was anticipated that the specific saturation magnetizations of the complexes would increase upon pyrolysis. Surprisingly, however, the increase in magnetization after treating the complexes at 600-700° C. was significantly greater than expected, and this could not be attributed to merely weight loss.

Transmission electron micrographs of microtomed thin sections of the complexes heated at 700° C. showed both 8-10 nm particles (the original size), together with a substantial fraction of irregularly-shaped, 50-150 nm agglomerates within the carbonaceous matrix. This strongly suggested that some cobalt particle sintering had taken place during the heat treatment at 700° C. Interestingly, cross-sectional micrographs of cobalt-copolymer complexes that were pyrolyzed at 500° C. for four hours, then at 700° C. for four hours, revealed a majority of 8-10 nm particles together with some of the 50-150 nm agglomerates, and the particles seemed to have more spherical character as compared to those heated only at 700° C.

The high saturation magnetizations of the cobalt nanoparticle complexes have been retained after aging under ambient conditions for over a year. Since cobalt oxides are antiferromagetic, retention of the high magnetizations demonstrates the oxidative stability of these complexes. These cobalt complexes were milled in an intensive mill at 700 RPM in an aqueous 10 M KOH solution to obtain fine, oxidatively-stable particles. This milling process yields materials with a saturation magnetization of 90 emu g⁻¹ material (only about a 10% loss), and this has now been aged for over 100 days without any further loss in saturation magnetization. These observations suggest that the pyrolyzed phthalonitrile blocks afford a durable barrier preventing the oxidation of cobalt nanoparticles under ambient conditions.

Magnetic susceptometry measurements (using a SQUID based sensor) were conducted on a pyrolyzed sample to further elucidate its magnetic properties. Two different values for cobalt specific saturation magnetization were obtained, associated with two different elemental analyses following two different sample digestion procedures (discussed in the following section and denoted D1 for 4 day digestion procedure and D2 for 13 day digestion procedure). Sample preparation for the first analysis consisted of digesting a known amount of sample in 35 mL (total volume over digestion period) of 1:1 HNO₃:H₂SO₄. The sample was digested while heating (70-100° C.) for 4 days. The sample for the second analysis was subjected to 35 mL (total volume over digestion period) of 1:1 HNO₃:H₂SO₄ for 13 days with an addition of 5 mL H₂SO₄ on the ninth day. The sample was heated (70-100° C.) during the entire digestion process. The protective graphitic coating discussed in (TEM section) is likely the cause for disagreement between elemental analysis data. Graphite is an extremely stable and unreactive allotrope of carbon. Such a coating would passivate a metallic nanoparticle, protecting it from chemical reactions (e.g. oxidation). The more rigorous digestion for the second analysis resulted in higher cobalt concentrations being measured, suggesting that the protective coating does degrade over time under extreme conditions. Room temperature measurements showed apparent saturation magnetizations of 230 emu g⁻¹ Co for D1 and 172 emu g⁻¹ for D2. Both the magnitude of these values and their differences indicate that the sample digestion procedure for this material is critical to the value of cobalt concentration obtained from the ICP-AES measurement. The value of 230 emu g⁻¹ Co for D1 is significantly higher than the maximum cobalt specific magnetization expected based on the value for bulk cobalt and hence implies that the sample digestion procedure was incomplete. The reduction in the apparent cobalt specific magnetization with increased digestion time suggests that the digestion process may be very slow for this material. Low temperature magnetization measurements indicate similar saturation magnetizations. Field-cooled σ vs H measurements show magnetic hysteresis with negligible field bias relative to zero-field-cooled σ vs H measurements suggesting an absence of a cobalt oxide layer around the metallic cobalt particles. The long-term saturation magnetization stability confirms that the pyrolyzed carbonaceous cobalt complexes are oxidatively stable. Compared to the pre-pyrolyzed sample, the cobalt specific magnetization for the pyrolyzed sample saturates at high fields in both room temperature and low temperature studies indicating an absence of the paramagnetic component that was observed for the pre-pyrolysed sample. It is believed that residual carbonyl species evolve during the pyrolysis procedure, effectively decreasing or eliminating the amount of paramagnetic species in the sample. Field cooled and zero field cooled σ vs T measurements in conjunction with σ vs H measurements at room temperature suggest that both the pre-pyrolysed and pyrolysed samples consist of a combination of particles that are superparamagnetic and magnetically blocked at room temperature. Room temperature hysteresis loop measurements show that both samples exhibit magnetic remanence and coercivity (pre-pyrolyzed sample: H_{c}=410 Oe, Mᵣ=19 emu g⁻¹ Co; pyrolyzed sample: H_{c}=416 Oe, Mᵣ=34 emu g⁻¹ Co).

Transmission electron microscopy (TEM) was employed to study the structure and morphology of the pyrolyzed cobalt-polymer composites. The particle size distribution was quite broad and indicated that some sintering had taken place during the heat treatment procedure. This sintering resulted in complex crystalline particles, which have been difficult to classify as any of the known phases of cobalt (fcc, hcp or epsilon). A GATAN image filter (GIF) was used to confirm the elemental identity of the particles (images not shown) before conducting high-resolution transmission electron microscopy (HRTEM). The sample consisted of highly crystalline particles, which facilitated the imaging of lattice fringes and “graphitic” coatings. The Fourier transform of the image indicates that the particles are crystalline. The measured 3.3 Å spacing for the inner ring of the Fourier transform is consistent with literature values for the interlayer spacing of graphite (3.4 Å) (Terrones, M.; Grobert, N.; Olivares, J.; Zhang, J. P.; Terrones, H.; Kordatos, K.; Hsu, W. K.; Hare, J. P.; Townsend, P. D.; Prassides, K.; Cheatham, A. K.; Kroto, H. W.; Walton, D. R. M. Nature, 388, 52-55, 1997.). The “graphite” sheets follow the contour of the particle and may possibly act as the barrier that protects the particles against oxidation. Selected area electron diffraction (SAED) patterns were difficult to interpret owing to the imperfect nature of the crystalline cobalt particles. Numerous twin planes, stacking faults and other crystal defects lead to elaborate diffraction patterns. In spite of this difficulty, nano-beam electron diffraction (NBD) was used study the crystallinity of the cobalt particles. By following Kikuchi bands, diffraction patterns of various zone axes were readily obtained. The diffraction information from NBD in conjunction with Fourier transforms of several crystalline particle images provided adequate information to conclude that the sample is comprised of a mixture of cobalt crystals (fcc, hcp, epsilon and perhaps others).

This Example shows that poly(styrene-b-4-vinylphenoxyphthalonitrile) copolymers have been employed to form stable dispersions of magnetic cobalt nanoparticles ˜8-10-nm in diameter. These particles were converted to oxidatively stable entities with protective carbonaceous coatings by pyrolysis in inert atmospheres. The pyrolyzed complexes exhibited minimal hysteresis and have retained their high saturation magnetizations (˜95-100 emu g⁻¹) under ambient conditions for over a year.

While the invention has been described in terms of its preferred embodiments, those skilled in the art will recognize that the invention can be practiced with modification within the spirit and scope of the appended claims.

1. A phthalonitrile composition, comprising: poly(styrene-b-4-vinylphenoxyphthalonitrile).

2. The phthalonitrile composition of claim 1, wherein the poly(styrene-4-vinylphenoxyphthalonitrile) is complexed to a Group VIII metal.

3. The phthalonitrile composition of claim 2, wherein the metal is cobalt.

4. The phthalonitrile composition of claim 2, wherein the metal is iron.

5. The phthalonitrile composition of claim 1, wherein the composition is included in a cobalt ferrofluid.

6. Poly(styrene-b-4-vinylphenoxyphthalonitrile).

7. A composition selected from the group consisting of:
(a) a block or graft copolymer wherein at least one block contains phthalonitrile moieties;
(b) a poly(styrene-b-vinylphenoxyphthalonitrile) block copolymer comprising a polystyrene block and a polyvinylphenoxyphthalonitrile anchor block and having the following structure (1): 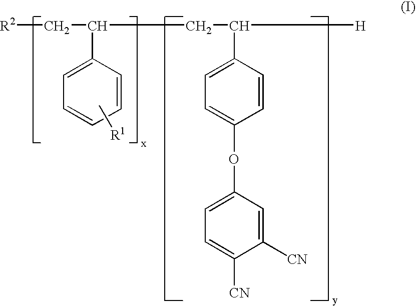
(wherein x and y denote the average numbers of repeating units in the block copolymer and each can vary between about 10-500; R¹ can be —H or an alkyl substituent having about 1-100 carbon atoms, aromatic substituents, or mixed alkyl aromatic substituents; R² can be any alkyl or aromatic groups derived from an initiator); or
(c) a graft copolymer comprising a random sequenced mixed anchor block and PDMS tail grafts, with the structure of the graft copolymer being according to the following formula (II): 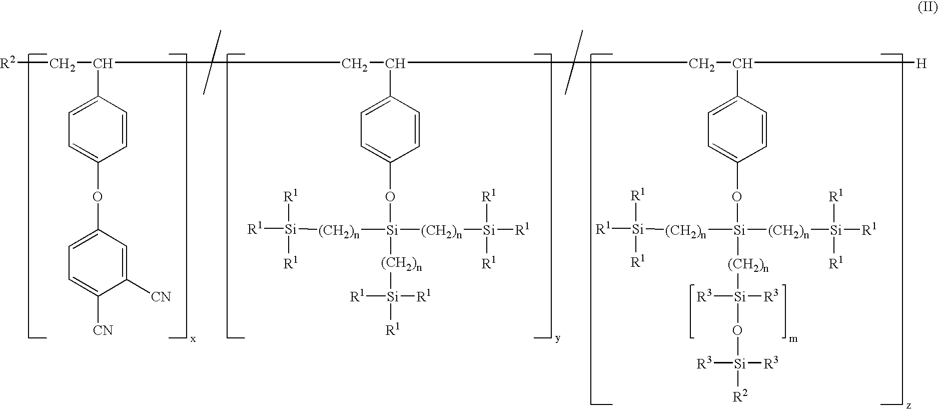
(wherein R¹ can be any group or combinations of groups that lead to a silica containing residue upon pyrolysis; R² can be any alkyl or aryl group derived from an initiator moiety; R³ can be alkyl groups containing about 1-6 carbons or aromatic groups; n is an integer of at least 1; m is an integer and represents an average number of repeating units in the polysiloxane grafts; x, y and z are each at least 1 and can be the same or different).

8. The composition of claim 7, wherein the structure of the graft copolymer is:

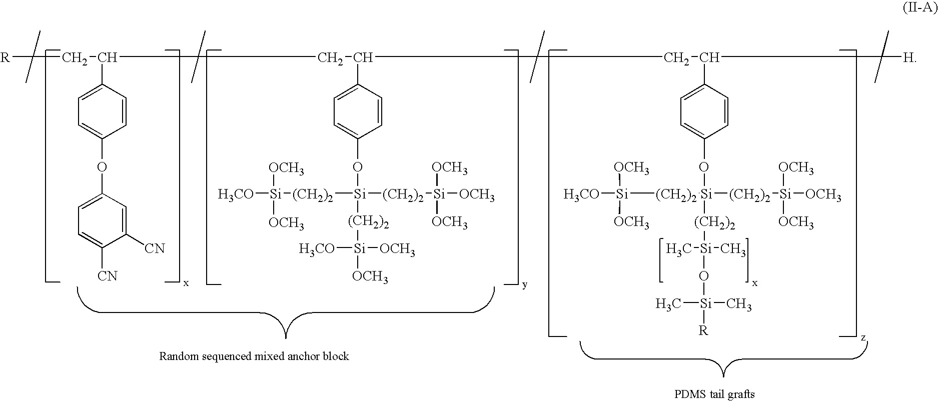

9. The composition of claim 7 wherein the block or graft copolymer is complexed to a group VIII metal.

10. The composition of claim 9 wherein the metal is cobalt or iron.

11. The composition of claim 10, wherein the composition is included in a cobalt ferrofluid or an iron ferrofluid.

12. The composition of claim 8, wherein the composition has been pyrolyzed at a temperature of 300° C. or greater to produce a pyrolyzed complex.

13. The composition of claim 12, wherein the pyrolyzed complex has a saturation magnetization of 50 emu g⁻¹ or greater

14. The composition of claim 12, wherein the pyrolyzed complex has a saturation magnetization of 90 emu g⁻¹ or greater.

15. The composition of claim 12, wherein the pyrolyzed complex has a surface functionalized with functional groups.

16. The composition of claim 15, wherein the functional groups are selected from the group consisting of amine; isocyanate; hydroxyl; polypeptides; polyglycolides; polylactides; poly(lactide-co-glycolides); poly(ethylene oxide); poly(ethylene oxide-co-propylene oxide); other polymers; streptavidin; avidin; other proteins; polynucleotides; vitamins; steroids; and other biospecific groups.

17. A polymer-metal complex, comprising:
a Group VIII metal complexed to a polymeric material that contains phthalonitrile groups.

18. The polymer-metal complex of claim 17, wherein the Group VIII metal is selected from the group consisting of Fe, Co, Ni, Ru, Rh, Pd, Os, Ir and Pt.

19. The polymer-metal complex of claim 17, wherein the Group VIII metal is cobalt or iron.

20. The polymer-metal complex of claim 17, wherein the polymeric material that contains phthalonitrile groups is poly(styrene-4-vinylphenoxyphthalonitrile).

21. The polymer-metal complex of claim 17, including a graft copolymer.

22. The polymer-metal complex of claim 17, wherein poly(styrene-4-vinylphenoxyphthalonitrile) is complexed to cobalt.

23. The polymer-metal complex of claim 17, wherein a saturation magnetization of the complex is at least 50 emu/g or greater.

24. The polymer-metal complex of claim 17, wherein the complex is carbon-encased.

25. A pyrolyzed matrix material, comprising:
a Group VIII metal; and
a carbonaceous matrix and optionally also a silica matrix, wherein the matrix comprises a pyrolysis product of a polymeric material containing phthalonitrile groups.

26. At least one highly magnetic nanoparticle, wherein the nanoparticle is encased in carbon, and the encased nanoparticle is highly magnetic having a saturation magnetization of at least about 50 emu/g.

27. The highly magnetic nanoparticle of claim 26, wherein the nanoparticle comprises:
a derivative of a phthalonitrile composition; and
a Group VIII metal.

28. The highly magnetic nanoparticle of claim 26, including poly(styrene-4-vinylphenoxyphthalonitrile) and cobalt.

29. The highly magnetic nanoparticles of claim 26, wherein the nanoparticles comprise a Group VIII metal.

30. The highly magnetic nanoparticles of claim 29, wherein the Group VIII metal is cobalt.

31. A method of making a styrene polymer, comprising at least the steps of:
(A) in a polystyrene compound containing a silyl ether bond, cleaving the silyl ether bond in a deprotection reaction to yield a deprotected polystyrene compound;
(B) reacting the deprotected polystyrene compound with a phthalonitrile compound, to yield a polystyrene polymer.

32. The method of claim 31, wherein poly(styrene-b-4-vinylphenoxyphthalonitrile) is made and the method includes:
(A′) cleaving the silyl ether bond of poly(styrene-b-tert-butyldimethylsilyloxystyrene) to yield poly(styrene-b-4-vinylphenol);
(B′) reacting the poly(styrene-b-4-vinylphenol) with 4-nitrophthalonitrile to yield poly(styrene-b-4-vinylphenoxyphthalonitrile).

33. A method of making a Group VIII metal ferrofluid, comprising at least the step of:
thermolysis of a reagent containing a Group VIII metal in a phthalonitrile solution.

34. The method of claim 33, wherein the group VIII metal is cobalt.

35. The method of claim 33, wherein the reagent containing a Group VIII metal is dicobalt octacarbonyl or iron pentacarbonyl.

36. The method of claim 33, wherein the phthalonitrile solution is a poly(styrene-b-4-vinylpheyxoyphthalonitrile) solution.

37. The method of claim 36, wherein the poly(styrene-b-4-vinylpheyxoyphthalonitrile) solution is concentrated.

38. The method of claim 33, including refluxing toluene in the presence of a poly(styrene-b-4-vinylphenoxyphthalonitrile) copolymer.

39. A method of making a carbon-encased, stable, magnetic nanoparticle, comprising at least the step of:
pyrolyzing Group VIII metal-nanoparticles to form carbon-encased nanoparticles.

40. The method of claim 39, wherein the Group VIII metal-nanoparticles are stabilized before the pyrolyzing step.

41. The method of claim 40, including a pre-pyrolyzing step of stabilizing Group VIII metal-nanoparticles with copolymers.

42. The method of claim 39, including concentrating ferrofluids to a solid state and pyrolyzing the concentrated ferrofluids.

43. The method of claim 39, wherein the pyrolyzing is at a temperature of about 700° C.

44. The method of claim 39, wherein the Group VIII metal-nanoparticles are cobalt nanoparticles.

45. The method of claim 41, wherein the copolymers in the stabilizing step are poly(styrene-b-4-vinylphenoxyphthalonitrile) copolymers.

46. A method of making a graphitic-encased nanoparticle which has functional groups on its surface, comprising at least the steps of:
(a) pyrolyzing Group VIII metal-nanoparticles to form carbon-encased nanoparticles;
(b) functionalizing the carbon-encased nanoparticles with functional groups to provide functional groups on a surface of the carbon-encased nanoparticles.

47. The method of claim 46, wherein the functional groups are selected from the group consisting of: amine; isocyanate; hydroxyl; polymers; proteins; polynucleotides; and biospecific groups.

48. The method of claim 47, wherein the functional groups are polymers selected from the group consisting of polypeptides, polyglycolides, polylactides, poly(lactide-co-glycolides), poly(ethylene oxide) and poly(ethylene oxide-co-propylene oxide).

49. The method of claim 47, wherein the functional groups are proteins selected from the group consisting of streptavidin and avidin.

50. The method of claim 47, wherein the functional groups are biospecific groups selected from the group consisting of vitamins and steroids.

51. A method of forming magnetic metal nanoparticles, comprising:
(a) preparing block copolymer dispersants for magnetic metals including at least one anchor block and at least one tail block, wherein the block copolymer comprises at least one block that is a precursor for a protective shell; followed by
(b) dispersing an organometallic metal precursor for a magnetic metal in a block copolymer solution and reacting with the block copolymer dispersants to form copolymer-magnetic metal nanoparticles; followed by
(c) heating the copolymer-magnetic metal nanoparticles to anneal the metal and form protective shells around the metal nanoparticles.

52. The method of claim 51, wherein the magnetic metal nanoparticles are protected with oxygen impermeable protective coatings.

53. The method of claim 51, including a step of refunctionalization to provide dispersibility or biospecific reactivity in biological fluids.
